(19) United States
(12) Patent Application Publication
(10) Pub. No.: US 2016/0320408 A1
(43) Pub. Date: Nov. 3, 2016

(54) METHODS OF T CELL EPITOPE PROFILING, MAKING T CELL COMPOSITIONS, AND TREATING DISEASES

(71) Applicant: OPEXA THERAPEUTICS, INC., The Woodlands, TX (US)

(72) Inventors: Don HEALEY, Anderson, TX (US); Lauren West COLLISON, The Woodlands, TX (US)

(21) Appl. No.: 15/105,995

(22) PCT Filed: Dec. 19, 2014
(86) PCT No.: PCT/US2014/071571

§ 371 Date: Jun. 17, 2016

Related U.S. Application Data
(60) Provisional application No. 61/918,570, filed on Dec. 19, 2013.

(51) Int. Cl. G01N 33/68; G01N 33/50; A61K 35/17; G01N 33/564

(52) U.S. Cl. CPC G01N 33/6866; G01N 33/564; G01N 33/505; A61K 35/17; G01N 2333/57; A61K 2035/124

(57) ABSTRACT

Disclosed herein is a method of detecting antigen specific T cells in a sample isolated from a subject and mapping immunostimulatory epitopes of the antigen. Such methods may be used in methods of making antigen specific T cell compositions, e.g., for the treatment of diseases such as cancer, infectious diseases and autoimmune disorders.

### FIELD OF INVENTION

The present invention provides methods of detecting antigen specific T cells in a sample, identifying the immunostimulatory epitopes of the antigen to which the antigen specific T cells respond, and use of same in making T cell compositions, e.g., for the treatment of disease.

### BACKGROUND OF THE INVENTION

The T cell response to an antigen involves recognition by T cells of fragments of the antigen, i.e., epitopes, which are presented in the context of antigen presenting molecules expressed on antigen presenting cells. Whether a given fragment of an antigenic protein is immunostimulatory epitope, i.e., capable of stimulating a T cell response, depends in large part on its binding properties to the antigen presenting molecule and the interactions of specific amino acids of the epitope with an appropriate T cell receptor. Detection of an antigen specific T cell and understanding which epitopes of an antigen participate in T cell mediated immunity, particularly during disease pathogenesis, provides a basis for directed modulation of the immune response and the development of vaccines and therapies against allergens, autoimmune diseases and tumors.

For example, in the case of autoimmune disease, immunotherapy with autologous T cells reactive against myelin protein epitopes has been demonstrated effective for depleting and/or negatively regulating myelin-reactive T cells and providing potential clinical benefit for patients suffering from multiple sclerosis (MS). However, T cell immunotherapy for each patient must be individualized because the T cell receptors of such autoreactive T cells are highly diverse and vary in their epitope specificity between different MS patients (Vandevyver et al., Eur. J. Immunol., 1995; 25:958-968, Wucherpfennig et al., J. Immunol., 1994; 152:5581-5592, Hong et al., J. Immunol., 1999; 163:3530-3538).

In addition to being individualized for each patient, the successful manufacture of T cell immunotherapies against autoimmune diseases requires the detection of autoreactive T cells and the identification of the epitopes to which the autoreactive T cells bind. The standard approach for mapping immunostimulatory epitopes of an antigen and cloning T cells for therapeutic use involves antigen priming, which generally involves incubation of T cells with the antigen, followed by plating individual cells into 96-well plates. Cells are then expanded and assayed for peptide specificities by screening clones with individual peptides which cover the antigen, a labor intensive and time consuming process. Peptide epitopes testing positive for immunostimulation are then used to obtain and expand clonal T cell lines for use in a T cell immunotherapy.

Cell therapy methods have also been developed to enhance the host immune response to tumors, viruses and bacterial pathogens. These cell therapy methods also often involve the ex-vivo activation and expansion of autologous T cells specific for antigens associated with the tumors or pathogens. Examples of these type of treatments include the use of tumor infiltrating lymphocyte (TIL) cells (see U.S. Pat. No. 5,126,132 issued to Rosenberg), cytotoxic T-cells (see U.S. Pat. No. 6,255,073 issued to Cai, et al.; and U.S. Pat. No. 5,846,827 issued to Celis, et al.), expanded tumor draining lymph node cells (see U.S. Pat. No. 6,251,385 issued to Tei man), and various other lymphocyte preparations (see U.S. Pat. No. 6,194,207 issued to Bell, et al.; U.S. Pat. No. 5,443,983 issued to Ochoa, et al.; U.S. Pat. No. 6,040,177 issued to Riddell, et al.; U.S. Pat. No. 5,766,920 issued to Babbitt, et al.).

However, the frequency of such antigen specific T cells in a sample easily obtained from the patient can be low. For example, the frequency of the autoreactive T cells in the peripheral blood of MS patients is approximately 1 in 10⁵ to 1 in 10⁶ peripheral blood mononuclear cells (Ota et al. (1990) Nature 346:183-7; Martin et al. (1990) J. Immunol. 145:540-8). This poses a challenge when screening a patient sample for autoreactive T cells during the manufacture of a T cell product since a typical yield from a 120 ml blood draw is about 100 million total T cells.

To this end, an assay is required that is sensitive enough to detect rare reactive T cells and identify the immunostimulatory epitopes to which they respond in a robust manner.

### SUMMARY OF INVENTION

Provided herein is a method for detecting antigen specific T cells in a sample and identifying the immunostimulatory epitopes of the antigen. As disclosed herein, a number of approaches were investigated to develop such an assay, resulting in the surprising finding that a macrobulk culture of T cells remains viable. Accordingly, a preferred assay format disclosed herein employs “macrobulk culture” of a sample comprising T cells, wherein the sample is cultured at a high concentration and density with epitope pools, wherein the pools preferably comprise overlapping peptides of an autoantigen.

Generally, the method of detecting an antigen specific T cell and identifying an immunostimulatory epitope to which the antigen specific T cell responds comprises (a) priming in vitro at least one macrobulk culture of a sample from a subject comprising T cells with an epitope pool comprising one or more peptides, wherein each peptide in the epitope pool is a distinct fragment of an antigen; (b) restimulating the macrobulk culture with the epitope pool for a period of time sufficient to allow for the detectable release of at least one activation cytokine by T cells specific for at least one of the peptides in the epitope pool; and (c) detecting the absence or presence of the at least one activation cytokine in said macrobulk culture; wherein the presence of the at least one activation cytokine in the culture detects a T cell specific for the antigen and identifies the region of the antigen spanned by the peptide(s) in the epitope pool as comprising an immunostimulatory epitope to which the antigen specific T cell responds. In one embodiment, multiple macrobulk cultures can be employed to further refine the results and allow for statistical analysis. In a preferred embodiment, an epitope pool comprises at least two peptides, each peptide shares a region of overlapping amino acid sequence identity with at least one other peptide in the epitope pool, and the peptides in the epitope pool together span a contiguous region of the antigen.

In one embodiment, the method comprises (a) priming in vitro each of a plurality of macrobulk cultures of samples from a subject comprising T cells with a distinct epitope pool from a library comprising at least two epitope pools, wherein each epitope pool in the library comprises one or more peptides that each comprises a distinct fragment of the antigen, (b) restimulating each macrobulk culture comprising T cells with the epitope pool with which it was primed for period of time sufficient to allow for the detectable release of at least one activation cytokine by activated T cells specific for at least one of the peptides in the epitope pool, and (c) detecting the absence or presence of an activation cytokine in each of the plurality of macrobulk cultures, wherein the presence of an activation cytokine in a macrobulk culture detects a T cell specific for the antigen and identifies the region of the antigen spanned by the peptide(s) in the epitope pool used to prime and restimulate the macrobulk culture as comprising an immunostimulatory epitope to which the T cell responds. In a preferred embodiment, an epitope pool comprises at least two peptides, each peptide shares a region of overlapping amino acid sequence identity with at least one other peptide in the epitope pool, and the peptides in the epitope pool together span a contiguous region of the antigen. In one embodiment, library of epitope pools comprises peptides that spans at least 1%, 5%, 10%, 20%, 30%, 40%, 50%, 60%, 70%, 80%, 90%, 95% or 99% of the antigen.

In one embodiment, a macrobulk culture comprises between about 2×10⁵ cell/mL/mm³ to about 2×10⁶ cell/mL/mm³. In one embodiment, a macrobulk culture comprises between about 4×10⁵ cell/mL/mm³ to about 1×10⁶ cell/mL/mm³. a preferred embodiment, a macrobulk culture comprises about 5×10⁵ cell/mL/mm³.

The sample is preferably obtained from a mammal. In one embodiment, the sample is obtained from a rodent. In a preferred embodiment, the sample is a human sample. In another embodiment, the sample further comprises antigen presenting molecules, which may be soluble or expressed by antigen presenting cells. In a preferred embodiment, the sample is a sample of peripheral blood mononuclear cells. In another preferred embodiment, the sample is human peripheral blood mononuclear cells.

In a preferred embodiment, the sample is obtained from a patient having a disease and the antigen is associated with the disease. In one embodiment, the disease is an infectious disease and the antigen is isolated from the infectious pathogen associated with the disease. In another embodiment, the disease is cancer and the antigen is a tumor associated or tumor specific antigen. In a preferred embodiment, the disease is an autoimmune disorder and the antigen is an autoantigen associated with the autoimmune disorder. Most preferably, the disease is multiple sclerosis and the antigen is a myelin protein. In one embodiment, the myelin protein is selected from the group consisting of myelin basic protein, proteolipid protein, myelin oligodendrocyte protein, and a combination thereof.

In certain embodiments, each peptide in a peptide pool is about 10 to about 20 amino acids in length, preferably about 16 amino acids in length. In another embodiment, each peptide pool comprises one or more peptides. In one embodiment, each peptide pool comprises at least two peptides, and the region of overlapping amino acids between the peptides is about 4 to about 16, preferably 12 amino acids in length

In one embodiment, the macrobulk culture is primed with the epitope pool for at least 1 to 10 days. In a preferred embodiment, the macrobulk culture is primed with the epitope pool for at least 5 days.

In one embodiment, the macrobulk culture is restimulated with the epitope pool for at least 12 hours in the presence of additional antigen presenting cells or peptide-loaded artificial APC. In a preferred embodiment, the macrobulk culture is restimulated with the epitope pool for at least 1 day.

In one embodiment, the activation cytokine is selected from the group consisting of IL-2, IL-4, IL-5, IL-9, IL-10, IL-13, IL-17, IL-18, IL-21, IL-22, IL-35,TNFα and IFNγ. In a preferred embodiment, the activation cytokine is IFNγ. In another embodiment, the detecting step comprises detecting the absence or presence of two activation cytokines, e.g., IFNγ and TNFα, IFNγ and IL-6, or TNFα and IL-6. In a preferred embodiment, the activation cytokine(s) is detected by a method selected from the group consisting flow cytometric analysis, enzyme linked immunosorbant assay (ELISA), bead based multiplex assay, and cytokine capture assay. Most preferably, the activation cytokine is detected by ELISA. In another embodiment, the activation cytokine(s) is detected via a bead based multiplex assay.

Also disclosed herein is a method of making a composition for the treatment of a disease comprising (a) detecting an antigen specific T cell in a patient having a disease and identifying an immunostimulatory epitope to which the antigen specific T cell responds according to the methods disclosed herein, wherein the sample comprising T cells is isolated from the patient and (b) propagating T cells isolated from the patient with the identified immunostimulatory epitope. In one embodiment, the disease is cancer and the antigen is a tumor associated or a tumor specific antigen for the cancer. Also disclosed herein are the compositions of T cells so made, and use of such compositions in methods of treating a disease, such as cancer. Such use comprises administering the composition comprising T cells in a therapeutically effective amount to a patient with cancer.

In one embodiment, the method of making a composition for the treatment of disease further comprises as a last step (c) attenuating the propagated T cells. In one embodiment, the disease is an autoimmune disorder, and the antigen is an autoantigen associated with the autoimmune disorder. In a preferred embodiment, the autoimmune disorder is multiple sclerosis and the autoantigen is selected from the group consisting of myelin basic protein, proteolipid protein, and myelin oligodendrocyte protein. Also disclosed herein are the compositions of T cells so made, and use of such compositions in methods of treating a disease, such as an autoimmune disorder. In a preferred embodiment, a composition comprising attenuated T cells made according to the method disclosed herein is used to treat an autoimmune disorder. Such use comprises administering the attenuated T cells in a therapeutically effective amount to a patient having an autoimmune disorder. In a preferred embodiment, the autoimmune disorder is multiple sclerosis and the autoantigen is selected from the group consisting of myelin basic protein, proteolipid protein, and myelin oligodendrocyte protein.

### BRIEF DESCRIPTION OF THE DRAWINGS

FIG. 1: CMV pp65 induced IFNγ response on day 4 with PBMC at two seeding densities

FIG. 2: CMVpp65 induced IFNγ response on day 6 with PBMC at two seeding densities

FIG. 3: Anti-myelin peptide IFNγ activity using MS donor 011034 PBMC as responder cells

FIG. 4: Anti-myelin peptide IFNγ activity using MS donor 011054 PBMC as responder cells

FIG. 5: Response to immunodominant Tetanus Toxin peptide epitopes direct ex vivo versus 5 days of microculture followed by IFNγ ELISpot. Data represent the mean and SD for quadruplicate ELISpot wells.

FIG. 6. Healthy donor 03106 anti-myelin peptide immunity detected on day 6 IFNγELlspot. Data represents mean and SD for quadruplicate ELISpot wells. CTRLs: negative controls.

FIG. 7: Healthy donor 03106 anti-myelin peptide immunity detected on day 6 by IFNγ ELISpot. Data represents mean and SD for quadruplicate ELISpot wells. CTRLs: negative controls.

FIG. 8: MS donor 03102 anti-myelin peptide immunity detected on day 6 by IFNγELISpot. Data represent mean and SD for quadruplicate ELISpot wells (Left panel assay 1, Right panel assay 2). CTRLs: cells in media alone.

FIG. 9: MS donor 03103 anti-myelin peptide immunity detected on day 6 by IFNγELISpot. Data represent mean and SD for quadruplicate ELISpot wells (Left panel assay 1, Right panel assay 2). CTRLs: cells in media alone.

FIG. 10: Impact of seeding density and a dissociation step on the detection of positive immunity to the MOGm16 peptide pool. Untreated: PBMC cultured in the absence of peptides in both micro-tubes and ELISpot assay. Data represents mean and SD for quadruplicate wells plated in the ELISpot assay.

FIG. 11: Shape and size distribution of ELISpot ‘spots’—inaccurate quantification of high frequency, hyper-reactive T cells. All 3 wells were counted using identical settings.

FIG. 12: MS Donor 03171—Comparison of ELISpot to cell ELISA for the detection of positive responses to myelin peptide pools. Cross-hatched bars represent cell ELISA data points greater than the upper limit of detection for the assay, and therefore represent positive responses without formal quantification. CTRL—control.

FIG. 13: MS Donor 03172—Comparison of ELISpot to cell ELISA for the detection of positive responses to myelin peptide pools. Cross-hatched bars represent cell ELISA data points greater than the upper limit of detection for the assay, and therefore represent positive responses without formal quantification. CTRL—control.

### DETAILED DESCRIPTION

The present invention provides methods of detecting antigen specific T cells in a sample and identifying the immunostimulatory epitope(s) of the antigen to which the T cells respond and/or are specific. Disclosed herein is the surprising discovery that macrobulk culture of immune cells, e.g., where the cells are cultured at a high density and in close proximity to each other, provides an environment conducive to the detectable activation of rare antigen specific immune cells in the culture when contacted with an immunostimulatory epitope. The methods disclosed herein generally include priming in vitro a macrobulk culture of a sample comprising T cells with an epitope pool comprising one or more peptides. The macrobulk culture comprising T cells is restimulated with the epitope pool to allow T cells specific for one or more peptides in the epitope pool to secrete detectable levels of activation cytokines. The detection of such activation cytokines correlates with the detection of an activated T cell in the sample and determination that the region of the antigen spanned by the peptides in the epitope pool comprises an immunostimulatory epitope for the activated T cell.

The T cells of the immune system recognize peptides complexed to antigen presenting molecules e.g., the major histocompatibility complex (MHC) in rodents or the human leukocyte antigen (HLA) in humans, expressed on antigen presenting cells (APCs). The specificity of antigen recognition by T cells is defined by several parameters: 1) affinity of the T cell receptor to the peptide complexed to the antigen presenting molecules; 2) primary sequence of the antigenic peptide; and 3) synergistic effects of certain amino acid combinations within the antigenic peptide. It is generally thought that a high level of antigen specificity is a feature of T cell activation. Accordingly, an antigen specific T cell as used herein refers to a T cell activated by a specific antigen, or immunostimulatory epitope thereof.

“Epitope” as used herein includes any peptide fragment of an antigen capable of specific binding to a T cell receptor in association with antigen presenting molecules. Epitope determinants usually are chemically active surface groupings of molecules such as amino acids or sugar side chains and usually have specific three dimensional structural characteristics, as well as specific charge characteristics. “Immunostimulatory epitopes” as used herein include any peptide fragment of an antigen capable of not only specific binding to the immune cell receptor but also activating the immune cell, e.g., a T cell upon binding.

An ordinarily skilled artisan will recognize that activation of T cells often results in proliferation. Accordingly, both priming and restimulating a sample comprising T cells with the epitope pools described herein stimulates the proliferation of T cells.

“Priming” as used herein refers to the initial contact between an adaptive immune cell and its specific antigen. Accordingly, in vitro priming refers to the initial in vitro stimulation of T cells with an epitope. In a preferred embodiment, T cells are primed, e.g., contacted, incubated, cultured, etc., with a peptide pool for at least 24 hours, and preferably at least about 2 to 10 days. Most preferably, T cells are primed with a peptide pool for 5 days.

Once an immune cell is primed with an antigen (or epitope thereof), subsequent contact between the immune cell and the antigen may be referred to herein as “restimulation.” In preferred embodiment, T cells are restimulated, e.g., contacted, incubated, cultured, etc., with a peptide pool for at least 2 hours, and preferably at least 12 hours or longer, e.g., 24, 48 or 72 hours. Most preferably, T cells are restimulated with a peptide pool for about 1 day.

In another aspect of the invention, a sample comprising T cells isolated from a subject of interest is contacted with an immunostimulatory epitope identified according to a method described herein to propagate T cells specific for the antigen from which the epitope is derived. In one embodiment, the sample is contacted with the immunostimulatory epitope for about 3 to 14 days, and preferably at least 5 days. Most preferably, a sample is contacted with the immunostimulatory epitope for a period of time sufficient to provide a therapeutically effective amount of T cells.

Macrobulk Culture

In one aspect of the invention, a macrobulk culture of a sample comprising T cells is contacted with peptides, to allow for in vitro priming and/or restimulation of the T cells. “Macrobulk culture” as used herein refers to culturing cells at a high concentration and density. Such high concentration and density may be accomplished, e.g., using a small volume of culture media and a culture tube rather than a flat or u-bottom plate or flask. For example, in contrast to plating 1×10⁶ cell in 1 mL in a 24 well plate, such may be plated in a 1.5 mL culture tube to increase the density of the cells. In an exemplary embodiment, at least 1×10⁶ cells, preferably at least 2.5×10⁶ cells, and most preferably at least 3×10⁶ cells are suspended in at least 1 mL, preferably at least 1.5 mL of culture media and cultured in a small culture tube, such as a 1.5 mL culture tube, and most preferably a 5 mL culture tube.

In a preferred embodiment, macrobulk culture of a sample comprises about 2×10⁵ cell/mL/mm³ to about 2×10⁶ cell/mL/mm³. In one embodiment, a macrobulk culture comprises between about 4×10⁵ cell/mL/mm³ to about 1×10⁶ cell/mL/mm³. In a preferred embodiment, a macrobulk culture comprises about 5×10⁵ cell/mL/mm³. In addition to the use of macrobulk culture, standard techniques are used for cell culture as described herein (e.g., priming, restimulating, and propagating T cells with peptides).

Samples comprising T cells and/or T cells may be isolated from any mammal, e.g., rodents, humans and the like. In a preferred embodiment, samples are isolated from mammals having a disease or provide a model for human disease. In a more preferred embodiment, the sample is isolated from a human, and most preferably from a human suffering from a disease, such as but not limited to an infectious disease, cancer, or an autoimmune disorder.

Samples comprising T cells and/or T cells can be isolated as fresh samples from a mammal, from an in vitro culture of cells from a mammal, from a frozen sample of cells, and the like. Suitable samples can include, for example, blood, lymph, lymph nodes, spleen, liver, kidney, pancreas, tonsil, thymus, joints, synovia, and other tissues from which T cells may be derived. In a preferred embodiment, the samples comprising T cells are isolated as peripheral blood mononuclear cells (PBMC). PBMC may be partially purified, for example, by centrifugation (e.g., from a buffy coat), by density gradient centrifugation (e.g., through a Ficoll-Hypaque), by panning, affinity separation, cell sorting (e.g., using antibodies specific for one or more cell surface markers), and other techniques that provide enrichment of PBMC and/or T cells.

In one exemplary embodiment, PBMC are isolated from a blood sample by standard Ficoll-Hypaque method. The blood sample is treated with heparin and underlain with a Ficoll solution. Following centrifugation, the recovered cells can be washed, for example, in PBS or T cell culture medium (e.g., RPMI 1640 supplemented with 2 mM L-glutamine, 100 μg/ml penicillin/streptomycin, 1 mM sodium pyruvate and 15% pooled human serum; AIM-V; OpTimizer CTS, and the like). Using well-known techniques, the washed cells can be resuspended in cell culture medium and placed in a culture tube to form a macroculture as described herein. In a preferred embodiment, the methods disclosed herein comprise priming a macrobulk culture of peripheral blood mononuclear cells at a concentration and density of about 4.0×10⁵ PBMC/mL/mm³ to about 2×10⁶ PBMC/mL/mm³. In a preferred embodiment, the methods disclosed herein comprise priming a macrobulk culture of peripheral blood mononuclear cells comprising T cells at a concentration and density of about 4.5×10⁵ PBMC/mL/mm³.

Peptides, Epitope Pools and Libraries

As described above, the methods disclosed herein comprise incubating the macrobulk culture of a sample comprising T cells with peptides, e.g., which may be part of an epitope pool, during the priming and restimulation steps. Generally, the peptides can be from about 9 amino acids to about 20 amino acids, or more, in length. In a preferred embodiment, the peptides are about 16 amino acids. Each peptide in an epitope pool or epitope library may be a distinct fragment of an antigen, e.g., shares amino acid sequence identity with a contiguous fragment of an antigen. In one embodiment, each peptide shares at least 75%, e.g., about 80%, 90%, 95%, 97%, 98%, 99% or 100% sequence identity with a contiguous fragment of an antigen, said fragment may be at least 7 amino acids in length and is less than the full length of the antigen.

The peptides can be derived from any suitable antigen. In certain embodiments, the antigen is at least about 4 kilodaltons (kD), at least about 6 kD, or at least about 10 kD. Suitable antigens can include, for example, antigens derived from infectious agents, antigens associated with autoimmune disorders, tumor associated or tumor specific antigens associated with various cancers, and the like.

In exemplary embodiments, the antigen is a tumor associated or tumor specific antigen associated with a particular cancer, including, but not limited to cytokeratins, particularly cytokeratin 8, 18 and 19; epithelial membrane antigen (EMA); human embryonic antigen (HEA-125); human milk fat globules such as MBr1, MBr8, Ber-EP4,17-1A, C26 and T16; desmin; muscle-specific actin; placental alkaline phosphatase; beta-human chorionic gonadotropin; alpha-fetoprotein; prostate specific antigen (PSA); carcinoembryonic antigen of colon adenocarcinomas; HMB-45; chromagranin-A; synaptophysin, tyrosinase, etc.

In additional exemplary embodiments, the antigen is derived from a pathogen. Nonlimiting examples include herpes simplex-2 virus VP16, tetanus toxin, influenza hemagglutinin, HIV gag, Cytomegalovirus pp65, HBV surface antigen, and other envelope and coat proteins of virus etc.

In preferred embodiments, the antigen is an autoantigen associated with an autoimmune disease. Nonlimiting examples of autoantigens include myelin basic protein, proteolipid protein, myelin oligodendrocyte protein, aquaporin 4, platelet membrane glycoproteins IIb-IIIa and Ib-IX, insulin, proinsulin, glutamic acid decarboxylase (GAD), GAD65, GAD67, heat-shock protein 65 (hsp65), islet-cell antigen 69 (ICA69), islet cell antigen-related protein-tyrosine phosphatase (PTP), GM2-1 ganglioside, Tep69, an islet-cell protein tyrosine phosphatase and the 37-kDa autoantigen derived from it (including IA-2), phogrin, human chondrocyte glycoprotein-39, collagen, collagen type II, cartilage link protein, ezrin, radixin, moesin, mycobacterial heat shock protein 6, desmoglien, β-2-GPI, Ku (p70/p80) autoantigen or its 80-kd subunit protein, the nuclear autoantigens La (SS-B) and Ro (SS-A), proteasome β-type subunit C9, the centrosome autoantigen PCM-1, polymyositis-scleroderma autoantigen (PM-Scl), autoantigen CENP-A, U5, the nucleolar U3- and Th(7-2) ribonucleoproteins, the ribosomal protein L7, hPop1, a 36-kd protein from nuclear matrix antigen, thyroid peroxidase and the thyroid stimulating hormone receptor, the human TSH receptor, acetylcholine receptor, muscular receptor kinase, or any other suitable autoantigen.

For longer antigens, the overlapping peptides may be sorted into peptide pools to form a library of at least two peptide pools. Peptide pools generally comprise one or more peptides. In one embodiment, the library of epitope pools comprises peptides that together spans at least 1%, 10%, 20%, 30%, 40%, 50%, 60%, 70%, 80%, 90%, 95% or 99% of the antigen. In a preferred embodiment, the library of epitope pools comprises peptides that span at least 90%, and more preferably at least 95%, and most preferably at least 98% of the antigen.

The peptides in a peptide pool typically, but not necessarily, overlap (i.e., share a region of amino acid sequence identity) of between about two and about fifteen, or more, amino acid residues. In a preferred embodiment, the peptides of a peptide pool overlap by at least four amino acids. In another embodiment, the peptides of a peptide pool overlap by about 10 amino acids. In a preferred embodiment, overlapping peptides overlap by about 12 amino acids. For example, peptide “n” may be residues 1 to 16 of the antigen and peptide “n+1” can be residues 4 to 20 of the antigen, etc. The skilled artisan will appreciate, however, that the length of the peptides and the amount of residue overlap between peptides can vary, depending on the length of the antigen and/or region of interest, the degree of resolution required, and the like.

The criteria for sorting the peptides into pools can vary, as will be appreciated by the skilled artisan. For example, in one embodiment, pools are provided of at least about 2 or about 3 to about 8 overlapping peptides (e.g., spanning a contiguous region of the antigen). In one embodiment, pools of 2 overlapping peptides are provided. In preferred embodiment, pools of at least 6 overlapping peptides are provided.

In other embodiments, the peptides are sorted into pools according to any other suitable criteria, such that the peptides in each pool are known or can be determined. In one embodiment, peptides are sorted into pools such that the peptides in a pool together span a distinct and contiguous region of the antigen. In another embodiment, the peptides are additionally sorted not only such that the peptides in a pool together span a distinct and contiguous region of the antigen, but also such that at least one peptide in a first pool shares a region of overlapping amino acid sequence identity with at least one peptide in a second pool and the peptides in the first and second pools together span a contiguous region of the antigen comprising both the contiguous region of the antigen spanned by the peptides in the first pool and the contiguous region of the antigen spanned by the peptides in the second pool.

In any of the embodiments, the peptides can be prepared in a variety of ways. For example, peptides can be synthesized using an automated peptide synthesizer. The peptides can also be manually synthesized. Alternatively, peptides can be generated by proteolytic cleavage (e.g., by trypsin, chymotrypsin, papain, V8 protease, and the like) or specific chemical cleavage (e.g., by cyanogen bromide). The peptides also can be synthesized by expression of overlapping nucleic acid sequences in vivo or in vitro, each nucleic acid sequence encoding a particular peptide.

The peptides optionally can be isolated and purified prior to contacting with the macrobulk culture of a sample comprising T cells. Suitable methods include, for example, chromatography (e.g., ion exchange chromatography, affinity chromatography, sizing column chromatography, high pressure liquid chromatography, and the like), centrifugation, differential solubility, or by any other suitable technique for the purification of peptides or proteins. In certain embodiments, the peptides can be labeled (e.g., with a radioactive label, a luminescent label, a chemi-luminescent label, an affinity tag, and the like) to facilitate purification of the peptides.

In another aspect, methods are provided for identifying candidate immunostimulatory epitopes. In certain embodiments, candidate immunostimulatory epitopes can be identified using a computer-implemented algorithm for candidate epitope identification. Such computer programs include, for example, the TEPITOPE program (see, e.g., Hammer et al., Adv. Immunol 66:67 100 (1997); Sturniolo et al., Nat. Biotechnol. 17:555 61 (1999); Manici et al., J Exp. Med. 189:871 76 (1999); de Lalla et al., J. Immunol. 163:1725 29 (1999); Cochlovius et al., J. Immunol. 165:4731 41 (2000); the disclosures of which are incorporated by reference herein), as well as other computer implemented algorithms.

The computer-implemented algorithm for candidate epitope identification can identify candidate epitopes in, for example, a single protein, in a very large protein, in a group of related proteins (e.g., homologs, orthologs, or polymorphic variants), in mixtures of unrelated proteins, in proteins of a tissue or organ, or in a proteome of an organism. Using this approach, it can be possible to interrogate complex tissues or organisms based on sequence information for expressed proteins (e.g., from a deduced open reading frame or a cDNA library), in addition to analysis of known candidate molecular targets, as an efficient, sensitive and specific approach to identification of potential T cell epitopes.

Following identification of candidate epitopes, peptides or pools of peptides can be formed that correspond to the candidate epitope(s). For example, once a candidate epitope is identified, overlapping peptides can be prepared that span the candidate epitope, or portions thereof, to confirm stimulation of T cells, and, as necessary, to refine the identification of that epitope. Alternatively, pools of peptides can be prepared including a plurality of candidate epitopes identified using a computer-implemented algorithm for candidate epitope identification.

Stimulating a Macrobulk Culture Comprising T Cells with Peptides

In another aspect, a macrobulk culture of a sample comprising T cells is contacted with a peptide pool to determine whether at least one of the peptides in the pool binds and stimulates the T cells in an epitope specific manner. Such contact may occur during priming, restimulation, and propagating steps. In some embodiments, multiple macrobulk cultures are used.

Generally, a macrobulk culture of a sample comprising T cells isolated from a subject of interest are cultured with peptides, which may be in a peptide pool that is part of a library of peptide pools. In a preferred embodiment, the macrobulk culture further comprises antigen presenting molecules, which may be soluble or expressed on cells, capable of presenting peptides in the correct context to the T cells isolated from a subject.

In some embodiments, the T cells are primed, restimulated, propagated, cultured, contacted, incubated, etc., for between about 1 to 10 days, or more, in T cell culture media in the presence of epitope pools or an immunostimulatory epitope to stimulate proliferation of T cells that are specific for the antigen from which epitopes are derived. The media optionally can be supplemented other components for the culture and/or viability of T cells (e.g., serum, antibiotics, cytokines, co-stimulatory receptor agonists, and the like).

The sample comprising T cells are contacted with the peptide pools/immunostimulatory epitope under suitable binding conditions. In one embodiment, the binding conditions are 37° C. in any suitable T cell culture media (e.g., RPMI 1640, AIM-V, OpTmizer CTS media), phosphate buffered saline, Dulbecco's phosphate buffered saline, Dulbecco's Modified Eagle Medium, Iscove's medium, and the like. The media can be supplemented with other components for the culture and/or viability of T cells (e.g., serum, antibiotics, cytokines, and the like). The appropriate concentration of peptides can be determined by titration. In one embodiment, each peptide in a peptide pool, or an immunostimulatory epitope, is added at a final concentration of about 2 ng/mL to about 100 μg/mL. In one embodiment, particularly for peptides that do not require further processing before presentation, the each peptide is added at a concentration between 20 and 200 ng/mL. For larger peptides, each peptide is added at a concentration of about 10 μg/mL to about 50 μg/mL, most preferably 20 μg/mL.

Detection of an antigen specific T cell and identification of its immunostimulatory epitope

An antigen specific T cell and the immunostimulatory epitope to which it binds may be identified by detecting the activation of the T cells. By comparing the activation state of different macrobulk cultures of samples comprising T cells from a subject when contacted with different peptide pools, one or more peptide pools can be identified that contain an immunostimulatory epitope of the antigen. In one embodiment, the detection of activated T cells in a macrobulk culture identifies the region of the antigen spanned by the peptides in the epitope pool incubated with the macrobulk culture as comprising an immunostimulatory epitope for the activated T cells.

In certain embodiments, one or more additional rounds (or cycles) of screening are performed, in which individual peptides in the identified peptide pool(s) are used to screen a macrobulk culture of a sample comprising T cells. By analysis of the individual peptides, the immunostimulatory epitope(s) may be identified as a peptide or peptides, or to a portion of one or more peptides. In related embodiments, additional peptides optionally can be synthesized to further define the epitope(s). For example, truncated peptides can be prepared to refine the identification of the epitope.

T cell activation may be determined using well-known methods to detect and/or measure any of multiple standard activation criteria (e.g., measuring T cell proliferation, release of activation cytokines, expression of cell-surface activation markers, etc.). (See, e.g., Novak et al., J. Immunol. 166:6665 70 (2001); Kwok et al., J. Immunol. 164:4244 49 (2000); Fraser et al., Immunology Today 14:357 (1993); Novak et al., International Immunology 13:799 (2001); the disclosures of which are incorporated by reference herein.).

In a preferred embodiment, activation is determined by detecting the presence of well-known activation cytokines Non-limiting examples include IL-2, IL-4, IL-5, IL-9, IL-10, IL-13, IL-17, IL-18, IL-21, IL-22, IL-35,TNFα and IFNγ e.g., IL-2, IFNγ, TNFα, and the like. In a preferred embodiment, activation is determined by detecting the presence of a single activation cytokine, preferably IFNγ.

In another embodiment, activation is determined by detecting the presence of at least a second well-known activation cytokine Preferably, the absence or presence of a second activation cytokine is detected if the activation level of the first activation cytokine is below a threshold to be considered present. In such an embodiment, activation may be determined by detecting the presence of a first and second activation cytokine

The activation level of an activation cytokine may be determined by comparison to a negative control culture, e.g., a macrobulk culture of a sample incubated with a negative control peptide or no peptide. Generally, the activation level of an activation cytokine may be determined by comparing the concentration of an activation cytokine in the supernatant of a macrobulk culture primed and restimulated according to the methods disclosed herein against the concentration of the activation cytokine in the supernatant of a macrobulk culture of a sample incubated as a negative control. The activation level may be measured in terms of fold increase over the negative control, e.g., a 1.5 fold increase in concentration may be considered an activation level of 1.5, a 10 fold increase over the negative control may be considered an activation level of 10, etc. In one embodiment, an activation cytokine is determined to be present if it's activation level is at least about 1.2, e.g., about 1.5 about 1.8, about 2, about 2.5, about 5, about 7.5, and preferably at least about 10. In another embodiment, a first and second activation cytokine is determined to be present if the cumulative activation levels of the first activation cytokine and the second activation cytokine reaches at least about 1.2, e.g., about 1.5, about 1.8, about 2, about 2.5, about 5, about 7.5, and preferably at least about 10.

In exemplary embodiments, the absence of presence of activation cytokines is detected using enzyme-linked antibodies, e.g., enzyme-linked immunosorbent spot (ELISPOT) and enzyme-linked immunosorbent assay (ELISA). In a preferred embodiment, activation is determined using ELISA. In another preferred embodiment, the absence or presence of activation cytokines is detected using a bead based assay.

Compositions comprising activated T cells and methods of using same to treat disease

Upon detection of an antigen specific T cell in a subject and identification of the immunostimulatory epitope to which it responds, the immunostimulatory epitope may be used in methods of making compositions comprising T cells specific for the antigen, e.g., for use in treating disease.

Accordingly, provided herein is a method of making a composition comprising antigen specific T cells, the method comprising (a) detecting an antigen specific T cell in a patient having a disease and identifying an immunostimulatory epitope to which the antigen specific T cell responds according to the methods disclosed herein, wherein the sample comprising T cells is isolated from the patient and (b) propagating T cells isolated from the patient with the identified immunostimulatory epitope. In one embodiment, the T cells are propagated to provide a therapeutically effective amount of T cells for administration to the patient in need thereof.

“Therapeutically effective amount” or “effective amount” means the amount of a composition, compound, therapy, or course of treatment that, when administered to a subject for treating a disease, disorder, or condition, is sufficient to effect such treatment for the disease, disorder, or condition. The “therapeutically effective amount” will vary depending on the composition, the compound, the therapy, the course of treatment, the disease, disorder, or condition, and its severity and the age, weight, etc., of the subject to be treated. In another embodiment, the method further comprises as a last step, the step of attenuating the T cells.

Also provided herein are compositions comprising antigen specific T cells made according to the method described above. In one embodiment, the compositions comprise attenuated antigenic specific T cells. In a preferred embodiment, the compositions comprise antigen specific T cells, which may be attenuated, in a therapeutically effective amount to treat a disease selected from the group consisting of a cancer, an infectious disease and an autoimmune disorder.

A skilled artisan will recognize that such compositions may find particular usefulness in treating cancers and infectious diseases, and when the T cells are attenuated, autoimmune disorders. Such methods comprise administering to a patient in need thereof a therapeutically effective amount of a composition comprising antigen specific T cells as provided herein. For these compositions, the antigen specific T cells are detected and the immunostimulatory epitopes identified according to the methods described herein, wherein the antigen is associated with the disease. In preferred embodiments, the samples comprising T cells for the priming, restimulating and propagating steps are isolated from the patient to be treated such that the composition administered comprises autologous T cells.

In one embodiment, the patient has cancer and the immunostimulatory epitope identified according to the methods disclosed herein is derived from a tumor-associated or tumor-specific antigen associated with the cancer. Cancers of particular interest are those that present tumor-associated or tumor-specific antigens. Such antigens may be present in an abnormal context, at unusually high levels, or may be mutated forms. Autologous T cells specific for the tumor antigen may be administered as part of the host T cell response against the tumor cells.

Examples of tumor antigens are cytokeratins, particularly cytokeratin 8, 18 and 19, as an antigen for carcinomas. Epithelial membrane antigen (EMA), human embryonic antigen (HEA-125); human milk fat globules, MBr1, MBr8, Ber-EP4,17-1A, C26 and T16 are also known carcinoma antigens. Desmin and muscle-specific actin are antigens of myogenic sarcomas. Placental alkaline phosphatase, beta-human chorionic gonadotropin, and alpha-fetoprotein are antigens of trophoblastic and germ cell tumors. Prostate specific antigen is an antigen of prostatic carcinomas, carcinoembryonic antigen of colon adenocarcinomas. HMB-45 and tyrosinase are antigens associated with melanomas. Chromagranin-A and synaptophysin are antigens of neuroendocrine and neuroectodermal tumors. Of particular interest are aggressive tumors that form solid tumor masses having necrotic areas.

Many conventional cancer therapies, such as chemotherapy and radiation therapy, severely reduce lymphocyte populations. While the subject therapy may alleviate this immunosuppression to some extent, a preferred course of combined treatment will use such lymphotoxic therapies before or after the subject therapy.

The compositions described above may also be administered as part of the host response to pathogens. Infections with certain viruses become chronic when the host anti-viral mechanisms fail. Such infections can persist for many years or even the life-time of the infected host, and often cause serious disease. Chronic infections associated with significant morbidity and early death include those with two human hepatitis viruses, hepatitis B virus (HBV) and hepatitis C virus (HVC), which cause chronic hepatitis, cirrhosis and liver cancer. Other chronic viral infections in man include those with human retroviruses: human immunodeficiency viruses (HIV-1 and HIV-2) which cause AIDS and human T lymphotropic viruses (HTLV-1 and HTLV-2) which cause T cell leukemia and myelopathies. Infections with human herpes viruses including herpes simplex virus (HSV) types 1 and 2, Epstein Barr virus (EBV), cytomegalovirus (CMV) varicella-zoster virus (VZV) and human herpes virus 6 (HHV-6) are usually not eradicated by host mechanisms. Infection with other agents that replicate intracellularly, such as pathogenic protozoa, e.g. trypanosomes, malaria and toxoplasma gondii; bacteria, e.g. mycobacteria, salmonella and listeria; and fungi, e.g. candida; may also become chronic when host defense mechanisms fail to eliminate them.

The compositions disclosed herein may be administered to a patient suffering from such a chronic pathogen infection, wherein T cells are specific to identified immunostimulatory epitopes of antigens derived from the pathogen. A variety of such antigens are known in the art, and available by isolation of the pathogen or expression by recombinant methods. Examples include HIV gp 120, HBV surface antigen, envelope and coat proteins of viruses, etc.

When the compositions described above comprise attenuated T cells, such compositions may also be administered to a patient in need thereof as a T cell immunotherapy. In one embodiment, the patient has an autoimmune disorder and the immunostimulatory epitope identified according to the methods disclosed herein is derived from an autoantigen antigen associated with the autoimmune disorder.

Nonlimiting examples of autoimmune disorders include multiple sclerosis, rheumatoid arthritis, autoimmune uveoretinitis, diabetes, neuritis, polymyositis, psoriasis, vitiligo, Sjogren's syndrome, autoimmune pancreatitis, inflammatory bowel diseases (e.g., Crohn's disease and ulcerative colitis), celiac disease, glomerulonephritis, scleroderma, sarcoidosis, autoimmune thyroid diseases (e.g., Hashimoto's thyroiditis and Graves disease), myasthenia gravis, Addison's disease, pemphigus vulgaris, primary biliary cirrhosis, pernicious anemia, and systemic lupus erythematosis. In a preferred embodiment, the autoimmune disorder is multiple sclerosis.

Examples of autoantigens useful in expanding T-cells for immunotherapy of autoimmune disorders include but are not limited to, myelin proteins such as myelin basic protein, proteolipid protein (PLP), and myelin oligodendrocyte glycoprotein for multiple sclerosis.

For scleroderma, systemic sclerosis, and systemic lupus erythematosus, autoantigens include, for example, β-2-GPI, Ku (p70/p80) or its 80-kd subunit protein, the nuclear autoantigens La (SS-B) and Ro (SS-A), proteasome β-type subunit C9, the centrosome autoantigen PCM-1, polymyositis-scleroderma autoantigen (PM-Scl) autoantigen CENP-A, U5, the nucleolar U3- and Th(7-2) ribonucleoproteins, the ribosomal protein L7, hPop1, and a 36-kd protein from nuclear matrix antigen.

For autoimmune disorders of the skin, useful antigens include, but are not limited to, the 450 kD human epidermal autoantigen, the 230 kD and 180 kD bullous pemphigoid antigens, pemphigus foliaceus antigen (desmoglein 1), pemphigus vulgaris antigen (desmoglein 3), BPAg2, BPAg1, type VII collagen, a 168-kDa mucosal antigen in a subset of patients with cicatricial pemphigoid, and a 218-kd nuclear protein (218-kd Mi-2).

Autoantigens associated with insulin dependent diabetes mellitus include, but are not limited to, insulin, proinsulin, GAD65 and GAD67, heat-shock protein 65 (hsp65), islet-cell antigen 69 (ICA69), islet cell antigen-related protein-tyrosine phosphatase (PTP), GM2-1 ganglioside, glutamic acid decarboxylase (GAD), an islet cell antigen (ICA69), Tep69, an islet-cell protein tyrosine phosphatase and the 37-kDa autoantigen derived from it (including IA-2), and phogrin.

Autoantigens associated with rheumatoid arthritis include, but are not limited to human chondrocyte glycoprotein-39, collagen, collagen type II, cartilage link protein, ezrin, radixin, moesin, and mycobacterial heat shock protein 6.

Autoantigens associated with autoimmune thyroid disorders include as nonlimiting examples thyroid peroxidase and the thyroid stimulating hormone receptor, and the human TSH receptor.

Autoantigens associated with myasthenia gravis include, but are not limited to acetylcholine receptor and a muscular receptor kinase.

### EXAMPLES

### Example 1

Evaluation of culture environment and duration of culture on the induction of an antigen-specific T cell response; evaluation of IFNγ ELISpot as a high sensitivity method to detect rare antigen-specific T cells after microculture, and evaluation of conventional ELISA on day 6 of culture to detect antigen-specific immunity and establishment of the Epitope Profiling Assay.

### Example 1.1

### Materials and Methods

Tissue Culture Media

Complete OpTmizer CTS media (Life Technologies) was used throughout methods development, and supplemented with heat-inactivated pooled human AB serum (Valley Biomedical) at 2% by volume and L-Glutamine (Life Technologies) to a final concentration of 2 mM prior to use. Expiration of reconstituted media was set at 1 month, following the manufacturer's instructions.

Peripheral Blood Mononuclear Cells (PBMC)

Peripheral blood mononuclear cells (PBMC) were obtained from healthy donor apheresis products procured at Key Biologics, Memphis, Tenn. PBMC from MS donors were collected as 120 ml blood draws at the clinical site under and IRB approved Opexa protocol OP-BD-007. Subject recruitment, screening and blood draws were collected at the following two clinical sites: Dr Gazda, Integra Clinical Research, LLC, San Antonio, Tex., and Dr Fox, Central Texas Neurology Consultants, Round Rock, Tex. Archived PBMC obtained under Opexa protocol 2005.00, a Phase 2b clinical trial designated ‘TERMS’, were also utilized in early methods development.

PBMC enrichment from apheresis and whole blood was achieved by a 1:2 dilution of the source material in Phosphate Buffered Saline (PBS), and multiple 30 ml aliquots of the diluted product overlaid into 50 ml conical tubes, each containing 15 ml Ficoll Hypaque Premium (GE Healthcare). Ficoll gradients were centrifuged for 20 minutes at 800 g. Mononuclear cells were collected from the interface from each tube, pooled, diluted 1:5 in PBS, and PBMC washed by centrifugation at 300 g for 10 minutes. Cells were resuspended in 50 ml PBS, counted and utilized in methods development, or cryopreserved at 5×10⁷ or 2×10⁸ PBMC per ml in CS10 cryoprotectant (BioLife), using the manufacturer's instructions. Cells were maintained at 2-8° C. in a Coolbox (Biocision) prior to transfer to a Coolcell (Biocision) for rate controlled freezing overnight at −80° C. PBMC were transferred to vapor phase liquid nitrogen for long term storage.

Cryopreserved PBMC from whole blood or apheresis products were recovered from liquid nitrogen storage by rapid thawing of up to four vials of cells at 37° C. in a water bath, and dilution into 50 ml of OpTmizer CTS complete media. Cells were centrifuged at 250 g for 10 minutes prior to resuspension in 10 ml complete OpTmizer CTS media, then counted and used for downstream methods development.

Investigation of Micro-Culture Assays Employing a Cytomegalovirus (CMV) Recall Response

PBMC were collected from a pre-screened HLA-A2 positive, anti-CMV immunoglobulin reactive healthy donor by apheresis. An optimized HLA-A2 binding 9-mer peptide from CMVpp65 (NLVPMVATV) was utilized at a concentration of 200 ng/ml in complete OpTmizer CTS media supplemented with 20 IU/ml IL-2 (R&D systems) to stimulate PBMC (1×10⁶ per 350 ul, or 1×10⁶/ml) in 1 ml micro-tubes for either 3 or 5 days. Micro-cultures were re-stimulated by the addition of 200 ng/ml peptide on either day 3 or day 5 of culture. Subsequent to peptide-pulsing for 24 hours, supernatants were collected and assayed for IFNγ content by sandwich ELISA.

Investigation of Anti-Myelin Immunity in Micro-Cultures Using Archived MS Donor PBMC

In early development, immunity to a panel of 109 peptides comprising the sequences of MBP, MOG and PLP was studied using archived PBMC from the TERMS clinical trial. The peptide library was constructed as a series of 16-mer peptides, with a 12-mer overlap. Peptides were mixed by mass in ‘pairs’ based on their linear position in the respective protein sequence for each myelin antigen. Sequence listings, paired mixes and final pools are provided as an appendix.

1×10⁶ PBMC per 1.2 ml round-bottomed micro-culture tube were established in 350 ul of complete OpTmizer CTS media supplemented with 20 IU/ml IL-2, and pulsed with 1 μg/ml of each peptide ‘pair’, totaling 55 micro-tube cultures. After 5 days, cultures were re-stimulated by addition of 1×10⁶ PBMC, and 1 μg/ml of the appropriate peptide pair. Negative control cultures received PBMC in the absence of peptide stimulation on days 0 and 5. On day 6, culture supernatants were harvested from all cultures and subjected to analysis of IFNγ content by sandwich ELISA.

Determination of IFNγ in Culture Supernatants by Sandwich ELISA

IFNγ content by sandwich ELISA was conducted using capture and detection antibodies with streptavidin-HRP from BD Bioscience, in addition to OptEIA Assay diluent. The ELISA was performed using the manufacturer's instructions. In brief, 96-well ELISA plates were coated with 100 ul of a 1:250 dilution of stock capture antibody in coating buffer (pH9.6) and incubated overnight at 2-8 C. Plates were washed 5 times with PBS using a Biotek ELx405 96-well plate washer. 100 ul of protein blocking solution was applied per well, and incubated at room temperature for 2 hrs. Blocking solution was removed by decanting the plates and blotting on absorbent tissue prior to addition of 100 ul volumes of supernatants to designated wells. In some experiments, supernatants were diluted from ‘neat’ to 1:8 in doubling dilution prior to addition to ELISA plates. In every case, the plate layout allowed for each plate to receive a standard curve of recombinant IFNγ to cover a range of 300 to 25 pg/ml. The LLOQ for the assay was set at 11.25 pg/ml. After 2 hrs of incubation, plates were washed 7 times with 0.05% Tween 20 in PBS prior to the addition of a 1:250 dilution of biotinylated detection antibody complexed to streptavidin-HRP. Plates were incubated for an additional hour at room temperature, then washed 14 times with 0.05% Tween 20 in PBS. 0-phenyldiamine (OPD) enzyme substrate was reconstituted by the addition of one urea tablet and one OPD tablet per 20 ml of distilled water. 100 ul of substrate solution was applied to all wells, and incubated for 30 minutes in the dark at room temperature. The optical density (OD) was measured using a BioTek ELx800 ELISA plate reader, with a filter set at 450 nm. Genie 5 software (BioTek) was utilized to generate the IFNγ standard curve, and to convert OD readings to concentrations of IFNγ (pg/ml).

Determination of the Frequency of IFNγ Secreting Cells by ELISpot.

All ELISpots were performed in a 96-well plate format utilizing reagents supplied in kit form by eBioscience. 24 hours prior to use, ELISpot plates were coated with 100 ul of a 1:250 dilution of anti-IFNγ capture antibody at 2-8 C. Capture antibody was decanted, and replaced with 200 ul of blocking solution consisting of RPMI media plus 10% FBS and plates further incubated at room temperature for 2 hrs. Blocking solution was decanted and PBMC preparations from various experiments (see experimental design and procedures) added to designated wells in a total volume of 100 ul. Where necessary, an additional 1×10⁵ PBMC were added to each well as a source of antigen presentation (APC) in a volume of 50 ul. Peptides were added at a concentration of 80 ug/ml in a volume of 50 ul to achieve a final peptide concentration of 20 ug/ml in the assay well. Where either APC or peptides were excluded (negative controls), media alone was substituted for volume of cell suspension or peptide solution as appropriate. For a positive control in the assay, control wells receiving responder cells and APC were pulsed with 50 ul of PHA-L at a concentration of 20 ug/ml in place of peptide. Loaded ELISpot plates were incubated at 37 C for approximately 18 hrs to allow the secretion and capture of IFNγ in the various microwells. On completion of cell culture, plates were removed from incubation and washed twice with distilled water to lyze and remove the cells, followed by a further five washes with 0.05% Tween 20 in PBS. All washes were performed using a BioTek ELx405 plate washer. To the washed plates was applied 100 ul per well of a 1:250 dilution of biotinylated anti-IFNγ detection antibody in assay diluent, and the plates further incubated at room temperature for 2 hrs. Plates were then washed again with seven changes of 0.05% Tween 20 in PBS, before addition of 100 ul per well of a 1:100 dilution of stock streptavidin-HRP in assay diluent. Plates were then incubated at room temperature for 1 hr. On completion, plates were washed seven times with 0.05% Tween 20 in PBS, followed by a further five washes in PBS alone. AEC substrate was prepared by the addition of 10 drops of the concentrate to 10 ml of assay diluent, and 100 ul of the substrate solution added to each ELISpot well. After a period of approximately 20 minutes, developing spots become visible, and the reaction is quenched by washing five times with distilled water. Developed plates were allowed to air dry over night prior to spot counting on a CTL Immunospot reader, using ImmunoSpot software version 5.1.8.

Quantification of anti-tetanus toxin (TT) responses by ELISpot

| | Peptide | | | Peptide | |
|---|---|---|---|---|---|
| Source | Designation | Amino Acid Sequence | Source | Designation | Amino Acid Sequence |
| CS Bio | TT1 | DIKNDLYEKTLNDYKAIANK | CS Bio | TT5 | LMQYIKANSKFIGITELKKL |
| CS Bio | TT2 | IVVYNLQSKITLPNDRTTPV | CS Bio | TT6 | GINGKAIHLVNNESSEVIV |
| CS Bio | TT3 | TNSVDDALINSTKIYSTFPS | CS Bio | TT7 | NNFTVSFWLRVPKSASHLE |
| CS Bio | TT4 | IDKISDVSTIVPYIGPALNI | | | |

Direct Ex Vivo ELISpot

PBMC from a healthy donor were directly seeded in quadruplicates at 2×10⁵ cells per well in 200 ul of complete Optmizer CTS media supplemented with 20 ug/ml of a single tetanus toxin peptide from a panel of seven (TT1-TT7) encompassing known immunodominant determinants. Peptides were synthesized using FMOC chemistry, and HPLC purified to greater than 95% peptide purity. Individual peptides were solubilized in dimethyl sulphoxide (DMSO), acetic acid and/or water to a final concentration of 5 mg/ml. Quadruplicate wells containing 200 ng/ml of the HLA-A2 CMVpp65 9-mer were included as a control recall antigen to which immunity to this peptide had been previously detected using PBMC from this donor. Negative control wells received PBMC in the absence of peptide. ELISpot plates were incubated overnight and subsequently processed for enumeration of IFN γ secreting cells by spot counting as described above.

ELISpot after 5 Days of Micro-Culture

PBMC were seeded at 1×10⁶ cells per micro-cluster tube in a volume of 500 ul of complete OpTmizer CTS media. Each tube was supplemented to 20 ug/ml of a single tetanus toxin peptide from a panel of seven (TT1-TT7) plus a final concentration of 5 IU/ml IL-2. Negative control micro-cultures consisted of cells in media plus IL-2, but in the absence of any peptide. After 5 days of culture, the contents of each microtube were gently resuspended and the culture distributed in 100 ul aliquots across four ELISpot wells to establish quadruplicate replica wells in the assay. Wells were additionally seeded with 1×10⁵ PBMC as a source of antigen presentation in a volume of 50 ul, and peptide overlayed at a concentration of 80 ug/ml in 50 ul to yield a final concentration of 20 ug/ml in the ELISpot cultures. Negative control wells again contained additional PBMC, but in the absence of peptide. After overnight incubation at 37 C, the ELISpot plates were developed and IFN γ spots enumerated as described above.

Quantification of Anti-Myelin Immunity Using Micro and Macro-Culture, and ELISpot Versus Cell-ELISA

A similar assay format as used for the quantification of a tetanus toxin peptide response after micro-culture was expanded to investigate anti-myelin immunity using 55 peptide ‘pairs’ encoding MBP, MOG and PLP as opposed to the seven peptide library utilized to detect the anti-tetanus response. In this format, 1×10⁶ PBMC in 500 ul culture volumes of complete OpTmizer CTS media supplemented with 5 IU/ml IL-2 plus a final concentration of 20 ug/ml of one peptide pair were established in a total of 55 micro-tubes to cover the complete myelin peptide library. Negative control cultures were again run in parallel composed of PBMC with IL-2, but in the absence of peptide.

To increase the total number of PBMC exposed to any one antigenic source, peptides were pooled equally based on mass in sets of six as opposed to testing in ‘pairs’, and 20 ug/ml of these peptide pools were added to 3×10⁶ PBMC in 1.5 ml of complete OpTmizer CTS in sterile 5 ml round-bottomed tubes on day 0.

On day 5 of culture, the cells in each micro-culture tube were gently resuspended, and 100 ul aliquots plated out in quadruplicate into ELISpot plates. For the macro-cultures in 5 ml tubes, 1 ml of media was removed and the cells resuspended in the remaining 500 ul prior to plating for assay. In every case, wells were restimulated with 1×10⁵ PBMC as a source of antigen presentation in the presence of 20 ug/ml of the matched myelin peptide pair or pool utilized on day 0. After overnight culture, the ELISpot plates were developed and IFN γ spots quantified. In some experiments, one half and one eighth of the culture was plated in duplicate wells each, as opposed to plating the whole culture evenly across four wells, so as to reduce the potential number of spots per well, thereby improving the accuracy of spot quantification.

As an alternative to ELISpot, cell-ELISA was investigated as a method to detect in situ cumulative IFNγ secretion, as opposed to the quantification of the number of IFNγ secreting cells. Day 5 cultures were seeded in the same manner as ELISpot wells. However in the cell-ELISA, ELISA plates previously coated with anti-IFNγ capture antibody substituted for ELISpot plates. After overnight incubation, the cells were removed from the plates by 5 washes with 0.05% Tween 20 in PBS, and the assay developed with reagents supplied by eBioscience, and used in the conventional sandwich ELISA described above. Optical densities were again converted to concentration of IFN γ by reference to a standard curve of IFN□ titered in the cell ELISA, but in the absence of cells.

In some experiments, a cellular ‘dissociation step’ was performed on the cultures prior to plating into ELISpot or cell-ELISA assays. This was achieved by centrifugation (10 minutes 250 g) of the culture tubes on day 5, removal of spent media, a subsequent wash with 0.5 ml of PBS, repeat centrifugation and then suspension in 0.5 ml of 1 mM EDTA, 10 U/ml DNAse in PBS for 10 minutes at 37° C. Dissociation solution was removed by centrifugation, and the cells replenished with 0.5 ml of complete OpTmizer CTS prior to downstream assay.

Optimized Epitope Profiling Assay

3×10⁶ PBMC are seeded in each of 18, 5 ml FACS tubes in 1.5 ml of complete OpTmizer media supplemented with 5 IU/ml IL-2. To each culture is added one of 18 peptide pools to a final concentration of 20 ug/ml. Two negative control tubes receive PBMC in media but in the absence of peptide. An additional tube is seeded with PBMC on day 0, and will be subsequently used as a positive control when pulsed with additional PBMC and PHA on day 5. All the tubes are loosely capped, and incubated at 37 C 5% CO₂ for 5 days. On day 5, 1 ml of spent media is removed from each tube, and 1×10⁶ PBMC added in a total volume of 0.5 ml supplemented with the matching peptide pool to achieve a final concentration of 20 ug/ml in a final culture volume of 1 ml. Negative control tubes receive 1×10⁶ PBMC in 0.5 ml media, but no peptide. The positive control (established from a tube that did not receive peptide on day 0) receives the additional 1×10⁶ PBMC with PHA-L substituted for peptide to achieve a final concentration of 2 ug/ml in a 1 ml final culture volume. Tubes are incubated at 37 C, 5% CO₂ for a period of 18-24 hrs. Supernatants are then harvested and doubly diluted from ‘neat’ to 1:8, and applied to a conventional sandwich ELISA. IFNγ concentrations are reported by reference to an IFN γ standard curve incorporated on each test plate.

A provisional definition of positivity for a ‘reactive’ peptide pool was defined by the IFN γ content in the 1:2 dilution of supernatant being greater than 2.5-fold above the negative control ie, PBMC cultures maintained in the absence of peptide throughout.

### Example 1.2

### Results

### Example 1.2.1

### Effect of Culture Environment and Duration of Culture on the Induction of an Antigen Specific T Cell Response

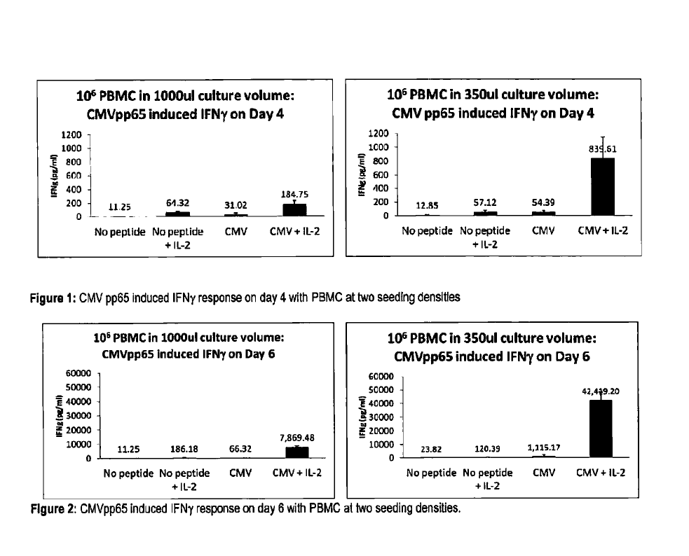

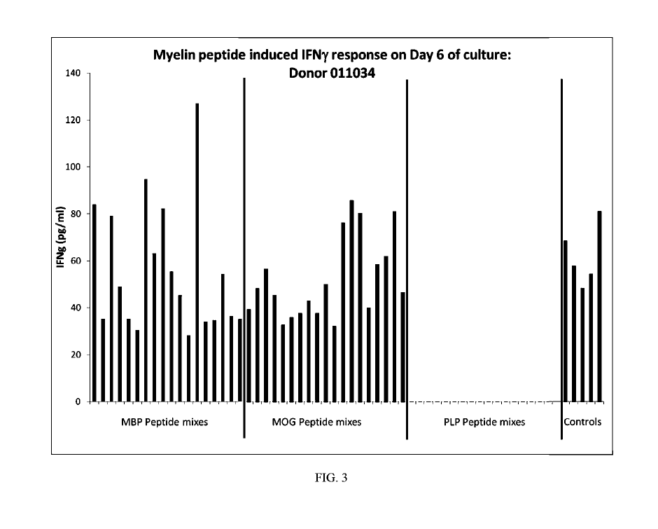

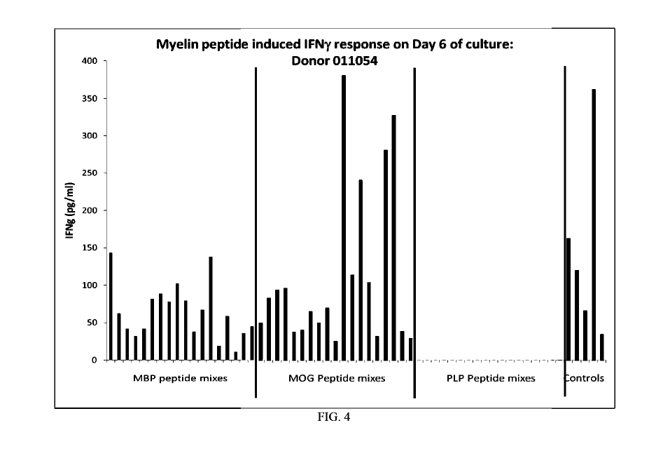

FIGS. 1 and 2 illustrate the impact of time and culture conditions on a CMVpp65 T-cell recall response, using an optimized 9-mer peptide presented by HLA-A2. Culturing cells at high density (1×10⁶ PBMC/350 ul) results in a 4-5 fold increase in the concentration of antigen-specific IFNγ detected at both day 4 and 6 of culture. The addition of 20 IU/ml IL-2 to the cultures greatly increases the IFNγ response by virtue of supporting T-cell proliferation, and thereby increasing the clonal representation of anti-CMVpp65 reactive T-cells in the cultures. A total of 6 days of culture, with antigen re-stimulation on day 5, results in a greatly increased anti-CMVpp65 T-cell response, compared to cells cultured and assayed after just 4 days in culture, with antigen re-stimulation on day 3.

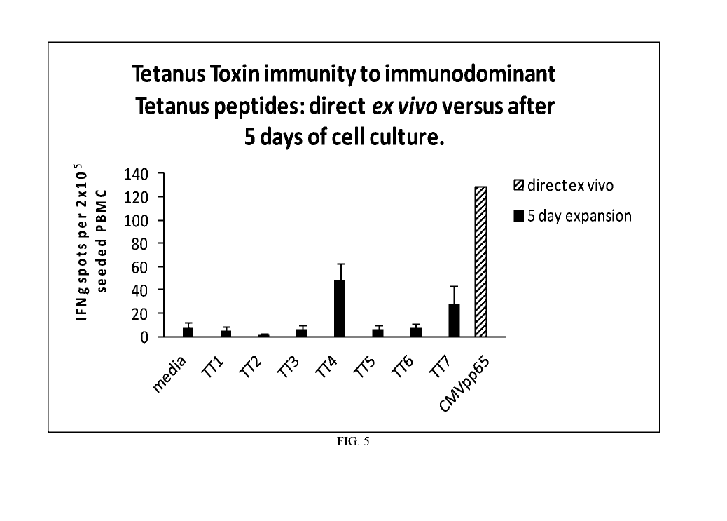

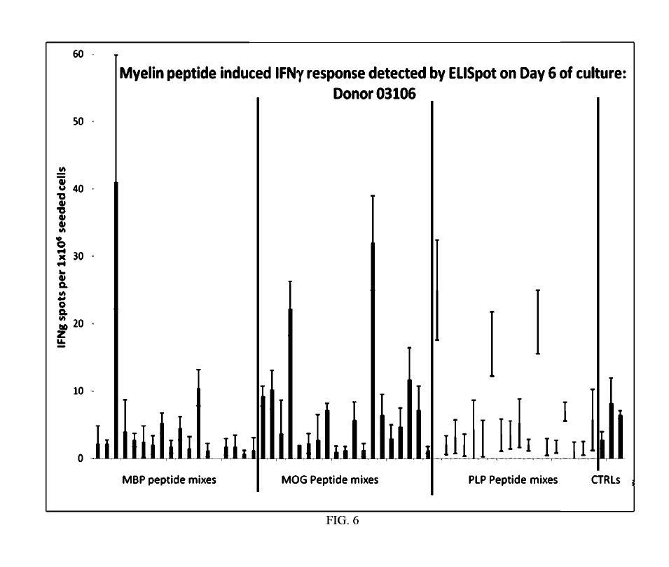

FIGS. 3 and 4 display the IFNγ responses of two MS donor PBMC preparations on stimulation with paired mixes of the 109 myelin peptide library using the seeding density, and culture conditions that generated optimal CMVpp65 antigen recall responses (FIG. 2). Both donors failed to show any significant IFNγ anti-myelin peptide activity over the negative control, media only micro-cultures, as defined by greater than 2-fold above background. The assay using donor 011054 shows the presence of ‘false-positive’ activity as defined by a high background response in one of the negative control cultures.

### Example 1.2.2

### Evaluation of IFNγ ELISpot as a High Sensitivity Method to Detect Rare Antigen-Specific T Cells after Microculture

The failure to observe clear anti-myelin peptide IFNγ activity suggested that either the culture conditions developed were insufficient to support a low frequency myelin-reactive T-cell repertoire, or that the assay readout was not showing sufficient sensitivity to delineate positive responses over background. To evaluate the latter, IFNγ ELISpot assays were implemented in place of the conventional sandwich ELISA to quantify the frequency of IFNγ secreting cells in the culture, as opposed to the cumulative IFNγ response measured in the supernatant. ELISpot assays have traditionally been employed to detect low frequency T-cell responses. In addition, in an effort to control background responses in the culture, the concentration of IL-2 was reduced from 20 IU/ml to 5 IU/ml.

To establish the impact of cell culture followed by ELISpot to quantify rare T-cell responses, an assay was setup whereby PBMCs from a healthy donor (03094) were subjected to direct ex vivo ELISpot against a panel of seven known immunodominant determinant peptides from Tetanus Toxin, as previously described in the literature. Alternatively, the assay was repeated on day 5 after cell culture in the presence of each peptide, and re-stimulation in the ELISpot environment.

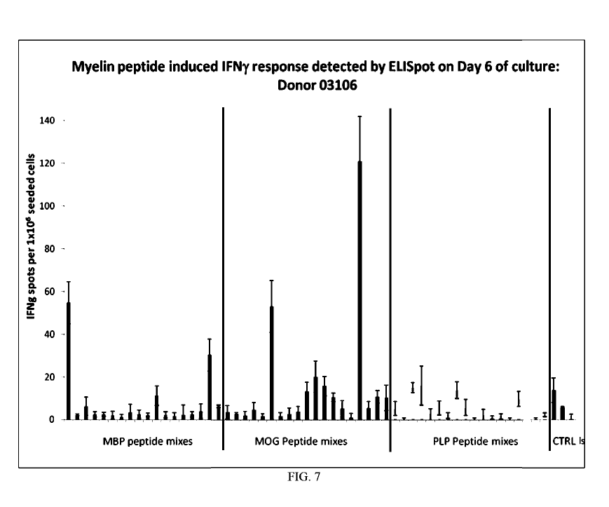

FIG. 5 shows that direct ex vivo ELISpot of the donor's PBMC failed to detect the presence of IFNγ secreting anti-tetanus toxin T-cells, although immunity to the HLA-A2 immunodominant peptide from CMV pp65 was readily detectable as a ‘recall’ response. However, when PBMC were subjected to 5 days of cell culture in the presence of each tetanus toxin peptide to expand out rare T-cell clones, and then followed by ELISpot, specific responses were obtained. To confirm that the response to at least TT4 was not a ‘false-positive’, a TT4-reactive T-cell line was successfully generated by weekly repetitive restimulation with peptide plus additional PBMC of a sample of donor cells over a 21 day culture period.

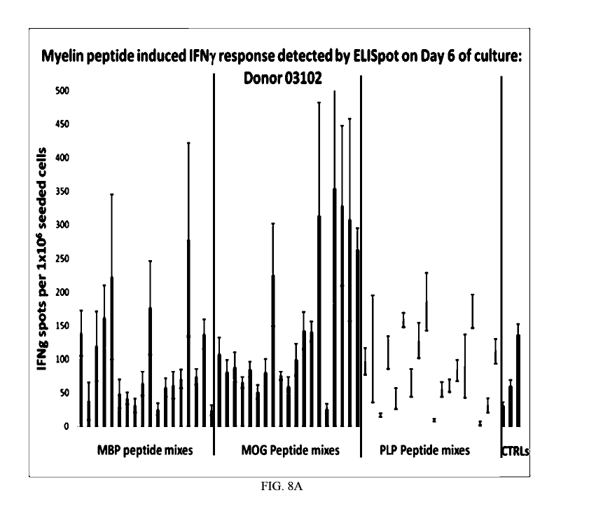

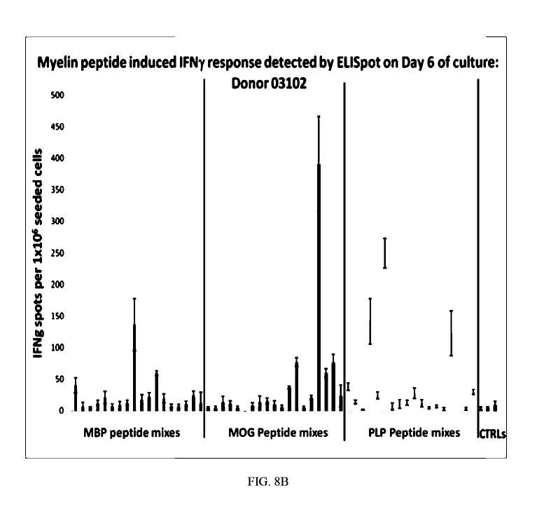

Having established the micro-cluster tube and ELISpot endpoint for a candidate antigen, namely Tetanus Toxin, and that micro-culture could amplify a tetanus response into a detectable range, the assay was carried forward for the investigation of anti-myelin T-cell immunity. The assay was first applied to healthy donor PBMC. Two assays were conducted from the same source archived tissue bank to reflect inter-assay variance. FIGS. 6 and 7 show inconsistencies in identifying peptide pools with positive responses across the two ELISpot assays.

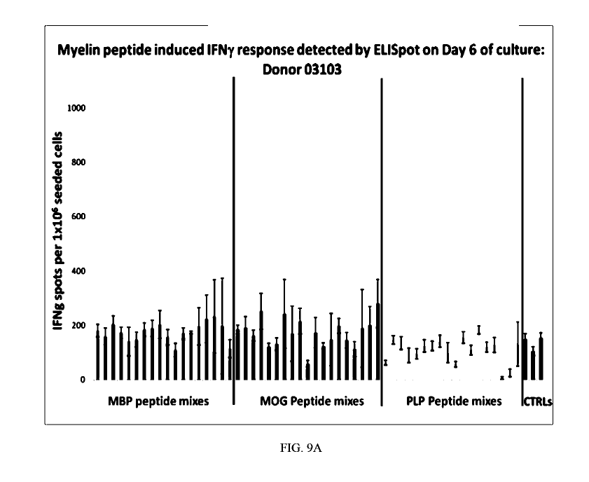

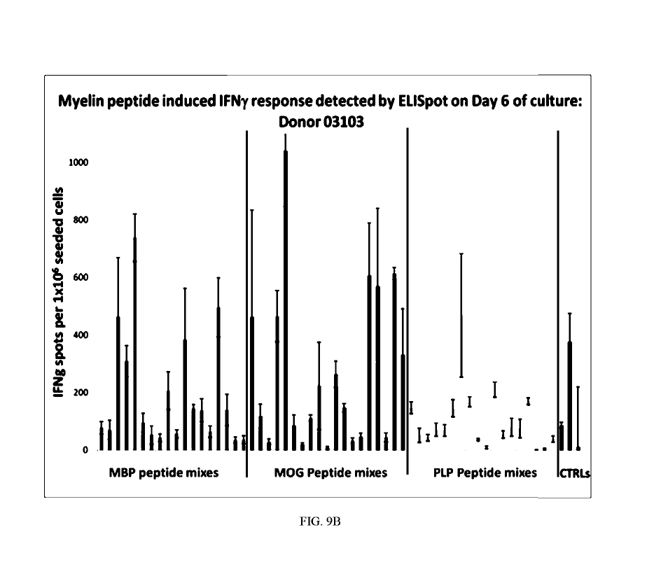

To confirm the poor inter-assay variance with the co-culture micro-cluster IFNγ ELISpot platform, the assay was applied to PBMC collected from two MS donors. The assay was performed twice per donor, the second assay utilized the same PBMC but from a cryopreserved source. Again, the data generated inconsistent results (FIGS. 8 and 9).

The lack of reproducibility, and the standard deviation across the quadruplicate wells for any one peptide mix suggested inefficient distribution of potentially reactive T-cells across each member well of a quadruplicate ELISpot series. This may be the result of aggregates forming in the micro-cluster tubes as a function of cell culture for 5 days that are inefficiently dissociated before transfer to ELISpot. Aggregates may also result in ‘false positive’ data in the ELISpot due to cellular debris being inefficiently removed from the ELISpot well on washing, followed by non-specific capture of the detection antibody resulting in ‘false spots’ in the assay. In an attempt to improve the distribution of cells across any one quadruplicate ELISpot set per peptide mix, and to reduce the risk of aggregates generating ‘false positive’ data points, the micro-culture samples were first subjected to a ‘dissociation step’ to increase the likelihood of plating out a single cell suspension, and reducing the number of cellular and debris aggregates carried over into the ELISpot assay. The dissociation step was achieved by washing the micro-cultures, and re-suspension in 1 mM EDTA with 10 U/ml DNAse in PBS for 10 minutes at 37° C., prior to re-suspension in media for cell plating and final analysis.

Another concern relates to the absolute frequency of anti-myelin T-cell immunity in terms of responder cells per number of PBMC sampled with each peptide pair. The use of the 109 peptide library mixed in ‘pairs’ dictated a PBMC sample size of 1×10⁶ cells per micro-culture, based on the likely yield of PBMC from a 120 ml blood draw to support the assay design. This sample size may be too small to expect to detect reliable anti-myelin T-cell immunity when the literature suggests that the frequencies of anti-myelin T-cells in peripheral blood may be in the range of 1 in 10⁵ to 1 in 10⁶ cells. To address this question, and in addition to the impact of aggregates on the final ELISpot assay, PBMC were setup in micro-cultures containing a PBMC sample size of either 1×10⁶, or 3×10⁶ cells in 350 ul volumes in micro-cluster tubes, and the response to the MOG peptide library was investigated, mixing MOG peptides in ‘pairs’ as previously described.

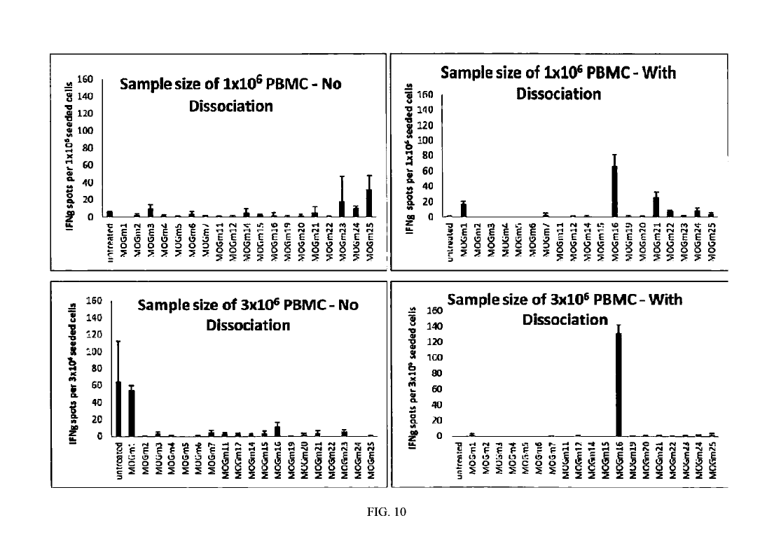

FIG. 10 shows that plating ELISpot from micro-tube cultures seeded with initially 1×10⁶ PBMC reveals a ‘weak’ response to peptide mix MOGm16. In this experiment, the application of the dissociation step had minimal impact on the potential for ‘false positives’ as defined by the ‘untreated’ media control. However, utilizing 3×10⁶ PBMC as day 0 seed material, results in a more robust response to peptide pool MOGm16, and the dissociation step removed the risk of ‘false positives’ as defined by the presence of spots in the untreated control.

The data above is supportive of increasing the sample size per peptide pool. However, with a finite number of PBMC to be recovered from a 120 ml blood draw, it is not possible to screen a 109 peptide library mixed in pairs, thereby generating 55 peptide ‘targets’ with a sample size of 3×10⁶ PBMC per target. To be able to increase the PBMC sample size to 3×10⁶ cells per peptide ‘target’ requires that the overlapping peptide library be mixed to generate ‘pools’ of 6 peptides, as opposed to just ‘pairs’. In reality, the MBP library consists of 6 ‘pools’ of 6 peptides. For MOG, 5 ‘pools’ of 6 peptides with the c-terminal pool (MOGp6) consisting of eight peptides. Finally, PLP consists of 5 ‘pools’ of 6 peptides, and one (PLPp6) consisting of 5 peptides. With this scenario, the peptide families covering MBP, MOG and PLP each consist of 6 ‘targets’ that encompass the full 109 peptide library. To facilitate the larger sample cell number per peptide pool, namely 3×10⁶ PBMC, cell cultures were established on day 0 in 1.5 ml volumes in 5 ml FACS tubes, as opposed to micro-cluster tubes in 350 ul volumes. This configuration was termed a ‘macrobulk’ culture.

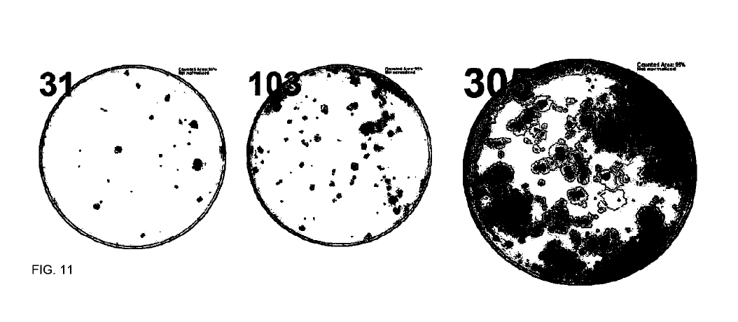

Another concern with the use of ELISpot was the ‘semi-quantitative’ nature of counting spots. Although the CTL Immunospot software can be calibrated to recognize spots of uniform nature, the spots formed in the ELISpot assays were very variable in terms of shape, size and distribution. This is best illustrated in FIG. 11. As the ELISpot assay was applied after a period of 5 days of co-culture, antigen-specific T-cells produce large spots of uneven size, reflecting their hyper-reactivity for IFNγ secretion, and their motility across the ELISpot well during the 18 hours of the assay on re-stimulation. The accuracy of counting spots degraded with increasing activity within the ELISpot well (FIG. 11).

To improve the accuracy of ELISpot counts, particularly having increased the seeding PBMC sample size from 1×10⁶ to 3×10⁶ PBMC on day 0 so as to increase the likelihood of detecting positive signals, ELISpot was setup with the inclusion of a cell dilution step so as to facilitate accurate spot counting. Briefly, the 1.5 ml day 5 cultures were centrifuged, spent media removed, the ‘cell dissociation’ step applied, and the cell pellet finally re-suspended in 400 ul of fresh media. Two wells of an ELISpot plate, representing one half of the macro-culture, were seeded with 100 ul each. The remainder of the cells (200 ul) was diluted further with 600 ul of fresh media before again 100 ul of cell suspension was distributed onto each of two ELISpot wells to create a 1:8 dilution of the original seeded macro-bulk culture.

As an alternative to counting the ‘number of cytokine secreting cells’ in ELISpot, a parallel cell ELISA was also established to detect ‘cumulative secretion’ of IFNγ. The format of the assay is essentially the same as for ELISpot, with the exception being that cells are plated out over anti-IFNγ antibody coated ELISA plates rather than nitrocellulose ELISpot plates. After restimulation by the addition of peptide pools and supporting PBMC as a source of APC, and 18 hours of culture, cells are washed away, and the plate developed as a conventional ELISA, including a colorometric substrate. The advantage of this approach is that the readout is truly quantitative when OD is interpreted against a concentration dependent standard curve of IFNγ. ELISpot and cell ELISA assays were established from the same cryopreserved PBMC source material, but each assay was initiated from day 0 on different days.

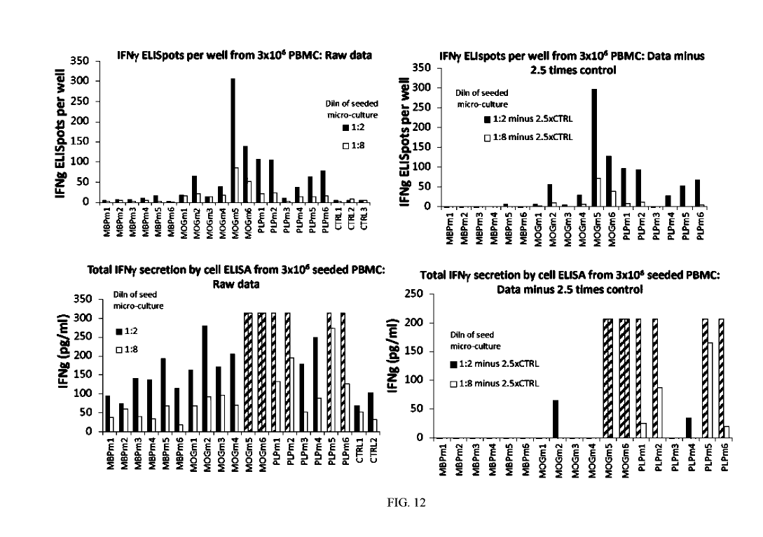

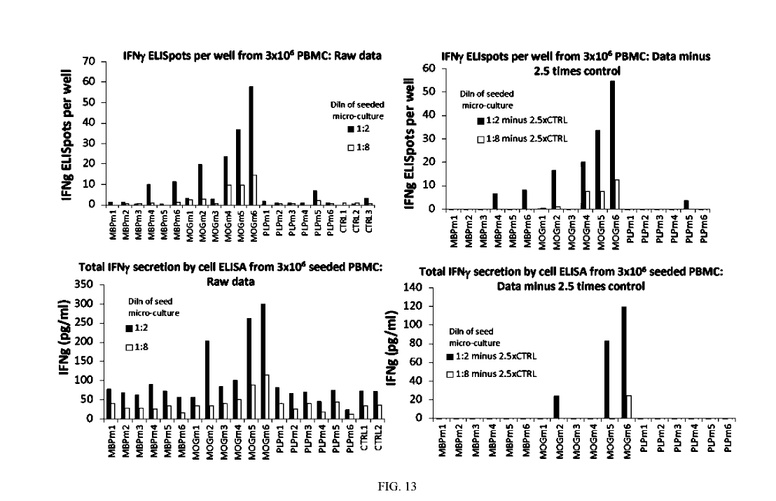

FIGS. 12 and 13 show that, for the first time in the development of the preferred Epitope Profiling Assay, concordance for the detection of responder peptide pools within an individual when performing independent assays based on the same cellular source material setup on different days, even when applying alternate assay platforms for the final readout. The improvement in the quality of the data is almost certainly related to the increase in initial culture size, namely 3×10⁶ versus 1×10⁶ PBMC on day 0. This increases the likelihood of capturing relatively rare myelin-reactive T-cells that can be expanded during the first 5 days of co-culture in the presence of any one peptide pool from the overlapping sequences that comprise the 109 peptide library.

Both the ELISpot and cell ELISA have issues with detectable range. ELISpot wells that contain high numbers of large, reactive spots are difficult to accurately quantify when the spot count exceeds 200. The cell ELISA has an upper limit of quantification of approximately 300 pg/ml that can result in some data points being ‘out-of-range’. To address these issues, both assays were setup including a dilution step of the seed material (FIGS. 12 and 13). To achieve accurate data requires precision dilution of a homogeneous cell suspension, which required the introduction of a cellular ‘dissociation step’, and then the distribution of ‘rare events’, namely myelin-reactive T-cells, evenly across the sample wells. Though achievable, the dilution series results in considerable manipulation of the macro-bulk-cultures, and their seeding across three 96-well plates in total, one for each antigen target, MBP, MOG and PLP. The complexity of performing cellular dilutions, and the ‘size’ of the assay becomes a challenge when the intent is to design a ‘high throughput’ platform.

### Example 1.2.3

### Evaluation of Conventional ELISA on Day 6 of Culture to Detect Antigen-Specific Immunity and Establishment of the Epitope Profiling Assay (EPA)

The readout for the cell ELISA is the concentration of IFNγ secreted from the cells, and thereby captured from the surrounding supernatant in situ within the assay itself. So rather than dilute the responder cell number to bring the analysis of IFNγ content within the range of the assay, a simpler approach is not to dilute the responder cells, but dilute the supernatant, and apply to a conventional ELISA as a secondary assay. As a homogenous cell suspension would then no longer be critical, the cellular ‘dissociation step’ can be avoided. On day 5, re-stimulation of the macro-bulk-cultures prior to collection of the supernatant for quantification of IFNγ content, can be conducted directly in the culture tube itself, significantly reducing the manipulation of the cultures.

As an initial test of the refined assay as stated above, a qualification was setup by four independent operators to detect immune responses from a healthy donor (3183) to published immunodomminant peptide sequences from tetanus toxin, encompassing a total of seven peptides (TT1-TT7). Briefly, each operator setup seven cultures of 3×10⁶ PBMC in 5 ml FACS tubes in 1.5 ml media volumes on day 0, and each pulsed with 20 ug/ml of one of the peptides from the panel of seven. On day 5, 1 ml of culture supernatant was removed, and 1×10⁶ PBMC were added as a source of antigen-presentation, and the cultures again pulsed with 20 ug/ml of peptide in a final culture volume of 1 ml. After overnight incubation, supernatants were collected and assayed for IFNγ content using a conventional ELISA with optical densities converted to pg/ml by reference to a standard curve of recombinant IFNγ. Supernatants were assayed ‘neat’ and diluted 1:2, 1:4 and 1:8.

Table 1 shows the concentration of IFNγ (pg/ml) measured above a threshold set at 2.5-fold over the negative control (PBMC cultured in the absence of peptide). The concordance rate reports the percentage of operators (four in total) who correctly identified positive or negative responses to individual peptide targets. Not surprisingly, where positive responses are detected, the concordance rate drops as the supernatant is titrated, reflecting differences in the absolute concentration of IFNγ detected between operators. Of importance, across the 4 operators, 100% concordance for positive activity was noted for TT peptides, TT3, TT5, TT6 and TT7 when assaying neat supernatants. 100% concordance for non-reactivity was noted for TT peptides, TT1 and TT2. Data for TT4 was more variable, with only one operator (HK) detecting a robust response. The lack of reproducibility to TT4 may reflect the detection of a rare primary immune response which may have been initiated on day 0 of culture.

TABLE 1
Inter-Operator qualification of an Epitope Profiling
Assay employing Tetanus Toxin peptides

| | | | | | | Concor- |
|---|---|---|---|---|---|---|
| | | | | | | dance |
| TT | Dilution of | Operators | | | | Rate |
| Peptide | Supernatant | CSA | HK | GM | MM | (%) |
| TT1 | Neat | 0 | 0 | 0 | 0 | 100 |
| | 1:2 | 0 | 0 | 0 | 0 | 100 |
| | 1:4 | 0 | 0 | 0 | 0 | 100 |
| | 1:8 | 0 | 0 | 0 | 0 | 100 |
| TT2 | Neat | 0 | 0 | 0 | 0 | 100 |
| | 1:2 | 0 | 0 | 0 | 0 | 100 |
| | 1:4 | 0 | 0 | 0 | 0 | 100 |
| | 1:8 | 0 | 0 | 0 | 0 | 100 |
| TT3 | Neat | ALOQ | ALOQ | 84 | 131 | 100 |
| | 1:2 | 184 | 138 | 44 | 30 | 100 |
| | 1:4 | 78 | 42 | 1 | 0 | 75 |
| | 1:8 | 14 | 0 | 0 | 0 | 75 |
| TT4 | Neat | 3 | 197 | 0 | 0 | 50 |
| | 1:2 | 0 | 66 | 0 | 0 | 75 |
| | 1:4 | 0 | 15 | 0 | 0 | 75 |
| | 1:8 | 0 | 0 | 0 | 0 | 100 |
| TT5 | Neat | ALOQ | ALOQ | ALOQ | ALOQ | 100 |
| | 1:2 | ALOQ | 226 | 137 | 235 | 100 |
| | 1:4 | 268 | 97 | 49 | 112 | 100 |
| | 1:8 | 120 | 32 | 6 | 66 | 100 |
| TT6 | Neat | 32 | 104 | ALOQ | 240 | 100 |
| | 1:2 | 0 | 21 | 257 | 101 | 75 |
| | 1:4 | 0 | 0 | 115 | 30 | 50 |
| | 1:8 | 0 | 0 | 39 | 15 | 50 |
| TT7 | Neat | ALOQ | 137 | ALOQ | 41 | 100 |
| | 1:2 | 140 | 40 | 132 | 0 | 75 |
| | 1:4 | 49 | 0 | 44 | 0 | 50 |
| | 1:8 | 5 | 0 | 5 | 0 | 50 |

Data represents IFNγ (pg/ml) over a positivity threshold set at 2.5-fold above the negative control (no peptide) cultures.
ALOQ: Above the Level of Quantification (313 pg/ml)

A further qualification of the EPA was conducted by an additional two operators, and using PBMC from a different donor (03190) with the tetanus toxin peptide panel. The data in Table 2 reflects the concentration of IFNγ (pg/ml) 2.5-fold above the negative control (no peptide) at the 1:2 dilution point of the test supernatants. The 1:2 dilution point was selected as the most appropriate titration point for analysis, as it required the signal to be titerable when compared to the signal detected in the ‘neat’ supernatant. Moreover, it would also exclude weak, and therefore possible false positive data, from being included in the analysis. The data shows 100% concordance between the two operators for positive reactivity to tetanus toxin peptides TT3, TT6, and TT7 for donor 03190.

TABLE 2
Secondary qualification of an Epitope Profiling
Assay employing Tetanus Toxin peptides

| | | | | | | | | Concor- |
|---|---|---|---|---|---|---|---|---|
| | | | | | | | | dance |
| | Tetanus Toxin Peptides | | | | | | | Rate |
| Operator | TT1 | TT2 | TT3 | TT4 | TT5 | TT6 | TT7 | (%) |
| LC | 0 | 0 | 37 | 0 | 0 | 26 | 60 | 100 |
| CA | 0 | 0 | 143 | 0 | 0 | 59 | 58 | |

Data represents IFNγ (pg/ml) over a positivity threshold set at 2.5-fold above the negative control (no peptide) cultures for the 1:2 dilution point of supernatants.

To qualify the preferred assay format with myelin peptide pools, 3×10⁶ PBMC were initiated on day 0 per peptide pool, with re-stimulation on day 5, and day 6 supernatants quantified for IFNγ content. Three repetitive EPAs per Operator were established using a single source of PBMC derived from a healthy donor. Table 3 shows that the donor (03190) had detectable responses to MOGp6, PLPp1 and PLPp4 when data was plotted from the 1:2 dilution point of the titered supernatants. The lower level of quantitation (LLOQ) of the IFNγ ELISA assay is 11.25 pg/ml. A positivity threshold was again set at 2.5-fold above the response in the no peptide control, which in these assays was equivalent to the LLOQ.

TABLE 3
Inter-operator and inter-assay qualification of the Epitope
Profiling Assay (EPA) detecting myelin-reactive T-cells.
Data represent the concentration of IFNγ (pg/ml) detected
above the positivity threshold in each culture.

| | | | | | | | Concor- |
|---|---|---|---|---|---|---|---|
| | Operator LC | | | Operator GM | | | dance |
| Peptide | As- | As- | As- | As- | As- | As- | Rate |
| mix | say 1 | say 2 | say 3 | say 1 | say 2 | say 3 | (%) |
| MBPp1 | 0 | 0 | 0 | 0 | 0 | 0 | 100 |
| MBPp2 | 0 | 0 | 0 | 0 | 0 | 0 | 100 |
| MBPp3 | 0 | 0 | 0 | 0 | 0 | 0 | 100 |
| MBPp4 | 0 | 0 | 0 | 0 | 0 | 0 | 100 |
| MBPp5 | 0 | 0 | 0 | 0 | 0 | 0 | 100 |
| MBPp6 | 0 | 0 | 0 | 0 | 0 | 0 | 100 |
| MOGp1 | 0 | 0 | 0 | 0 | 0 | 0 | 100 |
| MOGp2 | 0 | 0 | 0 | 0 | 0 | 0 | 100 |
| MOGp3 | 0 | 0 | 0 | 0 | 0 | 0 | 100 |
| MOGp4 | 0 | 0 | 0 | 0 | 0 | 0 | 100 |
| MOGp5 | 0 | 0 | 0 | 0 | 0 | 0 | 100 |
| MOGp6 | 40 | 14 | 0 | 5 | 67 | 55 | 83 |
| PLPp1 | 152 | 79 | 152 | 286 | 225 | 278 | 100 |
| PLPp2 | 0 | 0 | 0 | 0 | 0 | 0 | 100 |
| PLPp3 | 0 | 0 | 0 | 0 | 4 | 0 | 83 |
| PLPp4 | 58 | 37 | 22 | 262 | 285 | 281 | 100 |
| PLPp5 | 0 | 0 | 0 | 0 | 0 | 51 | 83 |
| PLPp6 | 0 | 0 | 0 | 0 | 0 | 0 | 100 |

The three immune reactive peptide pools that had repetitive positive scores for this donor were MOGp6, PLPp1 and PLPp4. The concordance rate for the two most reactive peptide pools (PLPp1 and PLPp4) was 100%. Reactivity to MOGp6 was weaker, and was detected in 5 of the 6 assays conducted. Therefore, there exists the possibility of false negative data, however, at low frequency, and only with peptide pools that are poorly reactive. On the basis of the data, fifteen peptide pools would be considered ‘non-reactive’. Across a total of 90 cultures (15 peptide pools over six assays), only two showed false positive data, representing a rate of approximately 2%. The ability to successfully generate myelin-reactive T-cell lines for use in T-cell immunotherapy protocols is dependent on only truly reactive peptide pools being selected for T-cell propagation. A very low false positive rate in the EPA greatly mitigates the risk of generating T-cell lines lacking antigen-specificity.

### Example 1.3

To further exemplify the use of cytokine detection to reveal positive T-cell immune reactivity to myelin peptides, supernatants from epitope profiling assays were subjected to a more detailed analysis. To this end, a bead-based multiplex assay was performed for the simultaneous detection of 13 cytokines to profile polyfunctional T-cell immunity to myelin peptide.

EPAs were performed on PBMC from 5 individual MS donors. As before supernatants, were collected on day 6, 24 hours after re-stimulation of cultures with additional PBMC and peptide.

Prior to multiplex assay, samples were centrifuged to remove cellular debris and stored at −20° C. until assay. In preparation for assay, reagents provided in the MILLIPLEX Magnetic Human Th17 kit, which include antibody-immobilized beads, quality control samples, and cytokine standards, were reconstituted in their appropriate buffers. Standard curve samples were prepared by performing 1:4 serial dilutions from the stock standard using the provided assay buffer. All wells of the provided plate were incubated for 10 minutes with 200 μL of assay buffer to prevent non-specific binding. Assay buffer was then discarded and 25 μL of each standard or control was added into the appropriate wells in addition to 25 μL of the appropriate matrix solution. To each sample well, 25 μL of assay buffer was added in conjunction with 25 μL of sample supernatant. All wells were then cultured with 25 μL of antibody-immobilized beads, protected from light, with agitation, for 18 hours at 4° C. Plates were then washed using a BioTek EL-405 plate washer and detection antibody solution applied for 1.5 hours. To the unwashed plate, the streptavidin PE solution was applied for 30 minutes and immediately washed to remove all excess reagents. All wells were then filled with 150 μL of MagPix running buffer prior to acquisition with the MagPix luminometer. Cytokine levels in samples was quantitated using the Millipore Milliplex Analyst software.

As seen in Table 4, all five donors showed positive reactivity to one or more peptide pools. In all cases, no peptide (NP) control cultures were used to set the baseline spontaneous levels of secretion of each cytokine Amongst the analytes detected, IFNγ, TNFα and IL-6 were most readily and most often detected.

TABLE 4
Cytokine levels of five MS donor samples in response to myelin peptides

| | | | | | | | | IL-12 | | | | | IL-17E/ | |
|---|---|---|---|---|---|---|---|---|---|---|---|---|---|---|
| Donor | Peptide | IL-27 | TNF-a | IL-10 | IL-5 | IL-17A | IL-6 | p70 | IL-23 | IL-17F | IL-2 | IL-4 | IL-25 | IFN-g |
| 14008005 | NP | <0.01 | 5.28 | 1.11 | <0.56 | <2.25 | 4.45 | 1.13 | <0.02 | <0 | <1.15 | <0.01 | <0.21 | 2.52 |
| | MOG2 | 0.02 | 20.11 | 3.05 | 0.85 | <2.25 | 181 | 1.13 | <0.02 | 0.01 | 36.44 | <0.01 | <0.21 | 111 |
| | MOG6 | 0.02 | 45.73 | 4.65 | 4.24 | 4.99 | 702 | 1.71 | <0.02 | 0.14 | 20.87 | <0.01 | <0.21 | 838 |
| 14008007 | NP | <0.01 | 5.28 | 2.02 | <0.56 | 2.54 | 9.52 | 1.13 | <0.02 | <0 | <1.15 | <0.01 | <0.21 | 2.52 |
| | PLP1 | 0.02 | 175 | 4.41 | 0.85 | 3.66 | 1285 | 1.71 | <0.02 | 0 | 6.3 | <0.01 | <0.21 | 222 |
| | PLP4 | 0.02 | 101 | 1.11 | <0.56 | <2.25 | 20.31 | 1.13 | <0.02 | <0 | 11.19 | <0.01 | <0.21 | 162 |
| 14002202 | NP | <0.01 | <1.11 | 1.11 | <0.56 | 3.28 | 89.79 | 1.41 | <0.02 | <0 | <1.15 | <0.01 | <0.21 | 3.17 |
| | PLP4 | 0.02 | 211 | 2.02 | 1.62 | 2.91 | 227 | 1.71 | <0.02 | 0.02 | 12.32 | 0.01 | <0.21 | 286 |
| 14012005 | NP | <0.01 | 7.11 | 3.27 | <0.56 | <2.25 | 99.67 | 0.99 | <0.02 | <0 | 1.28 | <0.01 | <0.21 | 5.27 |
| | MOG4 | 0.08 | 316 | 3.49 | 0.85 | 2.54 | 2693 | 1.71 | <0.02 | <0 | 1.67 | <0.01 | <0.21 | 816 |
| 14008003 | NP | 0.07 | 3.13 | 1.28 | <0.56 | 3.66 | 3045 | 2.03 | <0.02 | 0.01 | 7.06 | 0.01 | <0.21 | 260 |
| | MBP1 | 0.09 | 13.66 | 1.63 | <0.56 | 3.66 | 3569 | 2.36 | <0.02 | 0.01 | 8.19 | 0.01 | <0.21 | 878 |
| | MOG4 | 0.06 | 31.65 | 13.51 | <0.56 | 3.66 | >11518 | 2.36 | 0.03 | 0.02 | 5.16 | 0.01 | <0.21 | 745 |
| | PLP1 | 0.07 | 82.06 | 7.37 | <0.56 | 4.03 | >11518 | 3.26 | 0.06 | 0.07 | 5.54 | 0.01 | <0.21 | 692 |
| | PLP4 | 0.07 | 73.85 | 3.05 | 1.24 | 3.66 | 4949 | 2.71 | 0.03 | 0.03 | 12.69 | 0.01 | <0.21 | 1585 |

Having established the utility of IFNγ, TNFα and IL-6 as lead activation cytokine candidates for the detection of polyfunctional MRTC, 8 further MS donors were subjected to the triple analyte multiplex assay. The data are shown in Table 5.

TABLE 5
Cyotkine levels of eight MS donor samples in response to myelin peptides

| | | | | | |
|---|---|---|---|---|---|
| | | Conc. | Analyte | | |
| Rank | Peptide | (pg/ml) | (Rule) | Positivity by Additional Rule | Positivity Threshold Values (pg/mL) |

Subject UPN-USN 14010029-03855

| | | | | | | | | |
|---|---|---|---|---|---|---|---|---|
| 1 | MBPp6 | 291.7 | IFNg (R1) | IFNg (R2) | TNFa | IL-6 | IFNg | IFNg (R1) |
| 2 | MOGp6 | 106.48 | IFNg (R3) | | 67.6 | 215.15 | 48.85 | 97.7 |
| 3 | PLPp1 | 202.6 | TNFa (R4) | | | | | |

Subject UPN-USN 14010030-03856

| | | | | | | | |
|---|---|---|---|---|---|---|---|
| 1 | PLPp1 | 89.21 | TNFa (R4) | TNFa | IL-6 | IFNg | IFNg (R1) |
| | | | | 48.5 | 127.4 | 48.5 | 97 |

Subject UPN-USN 14016007-03823

| | | | | | | | | |
|---|---|---|---|---|---|---|---|---|
| 1 | MOGp4 | 149 | IFNg (R1) | IFNg (R2) IFNg (R3) TNFa (R4) | TNFa | IL-6 | IFNg | IFNg (R1) |
| 2 | PLPp1 | 129 | IFNg (R1) | IFNg (R2) IFNg (R2) TNFa (R4) | 82.35 | 510 | 56.75 | 113.5 |
| 3 | PLPp4 | 117 | IFNg (R1) | | | | | |

Subject UPN-USN 14023002-03824

| | | | | | | | | |
|---|---|---|---|---|---|---|---|---|
| 1 | MOGp3 | 5076 | IFNg (R1) | IFNg (R2) IFNg (R3) TNFa (R4) | TNFa | IL-6 | IFNg | IFNg (R1) |
| 2 | PLPp6 | 3882 | IFNg (R1) | IFNg (R2) IFNg (R3) TNFa (R4) | 174.8 | 200.05 | 324.2 | 648.4 |
| 3 | PLPp4 | 3499 | IFNg (R1) | IFNg (R2) | | | | |
| 4 | MOGp4 | 911 | IFNg (R1) | IFNg (R2) IFNg (R3) TNFa (R4) | | | | |
| 5 | PLPp5 | 327 | IFNg (R3) | | | | | |

Subject UPN-USN 14027005-03846

| | | | | | | | | |
|---|---|---|---|---|---|---|---|---|
| 1 | PLPp4 | 1680 | IFNg (R1) | IFNg (R2) IFNg (R3) TNFa (R4) | TNFa | IL-6 | IFNg | IFNg (R1) |
| 2 | PLPp3 | 368 | IFNg (R1) | IFNg (R2) IFNg (R3) TNFa (R4) | 48.5 | 85.95 | 48.5 | 97 |
| 3 | MOGp4 | 184 | IFNg (R1) | IFNg (R2) IFNg (R3) TNFa (R4) | | | | |
| 4 | PLPp1 | 94.35 | IFNg (R2) | IFNg (R2) TNFa (R4) | | | | |
| 5 | MOGp2 | 122 | IFNg (R1) | | | | | |

Subject UPN-USN 14102003-03831

| | | | | | | | |
|---|---|---|---|---|---|---|---|
| 1 | PLPp4 | 95.14 | IFNg (R1) | TNFa | IL-6 | IFNg | IFNg (R1) |
| 2 | PLPp6 | 54.82 | IFNg (R1) | 196.2 | 199.9 | 16.05 | 21.4 |
| 3 | MOGp3 | 28.14 | IFNg (R1) | | | | |
| 4 | MOGp6 | 21.96 | IFNg (R1) | | | | |

Subject UPN-USN 14016004-03874

| | | | | | | | |
|---|---|---|---|---|---|---|---|
| 1 | MOGp3 | 124 | IFNg (R1) | TNFa | IL-6 | IFNg | IFNg (R1) |
| 2 | PLPp1 | 58.52 | TNFa (R4) | 48.85 | 48.85 | 48.85 | 97.7 |

Subject UPN-USN 14007001-03890

| | | | | | | | | |
|---|---|---|---|---|---|---|---|---|
| 1 | PLPp6 | 238 | IFNg (R1) | IFNg (R2) IFNg (R3) TNFa (R4) | TNFa | IL-6 | IFNg | IFNg (R1) |
| | | | | | 49.6 | 73.8 | 48.85 | 97.7 |

### Example 1.4

### Discussion

The focus of the method development was to generate a reliable platform to detect myelin-reactive T-cells (MRTC) in PBMC with the final functional readout being the secretion of IFNγ.

Two primary considerations were addressed:

1) The low frequency of MRTC in PBMC, and its impact on sample size for the detection of positive immunity.

2) A preferred detection system that would enable a highthroughput readout of positive immunity to individual peptide pools.

The original intent was to use the 109 peptide library in pairs, creating 55 ‘targets’ against which T-cell immunity would be measured. However, the utilization of a 120 ml blood draw as source material will typically restrict the total number of PBMC available for assay to 120-150 million cells, comprising approximately 100-120 million T-cells. Simple ex vivo assays, such as ELISpot, were found to be too insensitive without a period of prior culture to expand out antigen-specific T-cells before assay. To utilize 55 peptide targets, and allowing sufficient cells to remain to re-stimulate cultures in the assay, required that a sample size of 1 million PBMC be assayed for immunity to any one peptide mix as ‘pairs’. Despite testing, an assay based on an initial sample size of 1 million PBMC could not be rendered robust. MRTC can be considered ‘rare events’ and with an incidence of between 1 in 10⁵ to 1 in 10⁶ T-cells amongst PBMC, a sample size of just 1 million PBMC per peptide mix or pool would be expected to be prone to ‘false negative’ data. To circumvent this limitation, peptides were pooled in groups of six, as opposed to pairs, creating a panel of 18 peptide targets with each evaluated against a PBMC sample size of three million cells in a ‘macro-bulk’ culture environment.

In total, three assay platforms were evaluated for the detection of positive anti-myelin immunity after a five day macro-bulkculture of PBMC in the presence of the 18 peptide pools encompassing MBP, MOG and PLP. ELISpot was considered the assay of choice, due to its potential to detect low frequency immune responses. However, employing the assay to detect immunity that had been pre-sensitized by 5 days of culture resulted in only semi-quantitative data. In addition the assay required dissociation of the cells prior to plating, and their titration to be able to reliably quantify ‘spots’. As an alternative, an in situ cell ELISA was developed to measure ‘cumulative’ IFNγ secretion. However, due to its lack of dynamic range, cells again required dissociation and titration to allow meaningful quantification of IFNγ. Although both assays did yield encouraging data, the complexity of the platform did not lend itself to a ‘highthroughput’ format.

The final assay configuration utilized a sample size of 3 million PBMC for each peptide pool on day 0, with re-stimulation of the cultures on day 5. Supernatants were harvested on day 6 and titered into a conventional IFNγ sandwich ELISA. As this approach avoided any need to titrate the responder cells, complexity of the assay platform was greatly reduced.

The preferred assay platform was evaluated for inter-operator, and inter-assay variance studying immunity to seven immunodominant peptides from tetanus toxin, in addition to immunity to the 18 myelin peptide pools.

An important consideration when designing the assay was to limit the likelihood of detecting ‘false positive’ data. To this end, a positivity threshold was initially set at 2.5-fold above the negative control cultures. In addition, this threshold was required to be breached with a 1:2 dilution of the culture supernatant within the ELISA. Choosing such a threshold would ensure that weak responses that could be scored positive only when analyzing data from the ‘neat’ supernatant could not be carried forward as potential ‘false positive’ peptide choices. With such a threshold, there remains the possibility that the assay may report the occasional ‘false negative’ (see Table 3). However, it's of the utmost importance that peptide pools that are selected represent robust responses so that T-cell lines can be generated successfully, and demonstrate antigen-specific immunity.

Definition of positive responses, and assignment of peptides for T-cell vaccine Manufacturing.

During development, a positive response to any one peptide pool was defined by the detection of a concentration of IFNγ (pg/ml) at the 1:2 dilution point of supernatant which is at least 2.5-fold higher than that recorded in the corresponding negative control cultures. If the concentration of IFNγ exceeds the upper level limit of detection of the assay (313 pg/ml), then the 1:4 and 1:8 dilution points must fall within range, and show evidence of titer. Utilizing the peptide pools that indicate the presence of IFNγ, e.g., having an activity level above a predetermined level, multiple T-cell lines can be generated for use as T-cell immunotherapies.

As development progressed, the utilization of a multiplex cytokine analysis of EPA supernatants, as opposed to the detection of a single analyte, e.g., IFNγ, allows the detection of a polyfunctional T-cell response to myelin peptide pools. As a consequence, a broader array of reactivity may be detected, resulting in more donors reporting positive reactivity to myelin. It is envisioned that detection of different combinations of cytokines may be useful for profiling epitopes involved in different diseases.

Table 5 displays the ‘activity level’ utilized to identify positive reactivity to myelin based on multipliers versus no peptide controls for each donor. To support the selection of positive reactivity to myelin peptide pools, the following activation levels determined the presence of one or more cytokines.

IFNγ response ALONE greater than 10× no peptide control. Ranked by IFNγ.

IFNγ response greater than 5× no peptide control AND TNFα response greater than 5× no peptide control. Ranked by IFNγ.

IFNγ response greater than 5× no peptide control AND IL-6 response greater than 5× no peptide control. Ranked by IFN γ.

TNFα response greater than 5×no peptide control AND IL-6 response greater than 5× no peptide control. Ranked by TNFα.

For each donor, Table 5 displays which how the presence of a cytokine, or a first and second cytokine, was determined for each positive peptide pool. The concentration (pg/ml) column lists the concentration of the highest ranked cytokine and its associated activity level. In many incidences, immunity to myelin peptide pools was determined by the presence of more than one cytokine. For seven of eight of the donors, MRTC reactivity was satisfied by the presence of an IFNγ. For one donor (14010030-03856) positivity was satisfied by the presence of both a TNF-α AND IL-6 signal but in the absence of IFNγ.

All patents and patent publications referred to herein are hereby incorporated by reference.

Certain modifications and improvements will occur to those skilled in the art upon a reading of the foregoing description. It should be understood that all such modifications and improvements have been deleted herein for the sake of conciseness and readability but are properly within the scope of the following claims

Myelin peptide sequences, mixes and pools

MBP

| Pool | Mix | Seq ID | Sequence |
|---|---|---|---|
| MBPp1 | MBPm1 | MBP 3 | QRHGSKYLATASTMDH |
| | | MBP 4 | SKYLATASTMDHARHG |
| | MBPm2 | MBP 5 | ATASTMDHARHGFLPR |
| | | MBP 6 | TMDHARHGFLPRHRDT |
| | MBPm3 | MBP 7 | ARHGFLPRHRDTGILD |
| | | MBP 8 | FLPRHRDTGILDSIGR |
| MBPp2 | MBPm4 | MBP 11 | SIGRFFGGDRGAPKRG |
| | | MBP 12 | FFGGDRGAPKRGSGKV |
| | MBPm5 | MBP 13 | DRGAPKRGSGKVPWLK |
| | | MBP 14 | PKRGSGKVPWLKPGRS |
| | MBPm6 | MBP 15 | SGKVPWLKPGRSPLPS |
| | | MBP 16 | PWLKPGRSFLPSHARS |
| MBPp3 | MBPm7 | MBP 17 | PGRSPLPSHARSQPGL |
| | | MBP 18 | PLPSHARSQPGLCNMY |
| | MBPm8 | MBP 19 | HARSQPGLCNMYKDSH |
| | | MBP 20 | QPGLCNVYKDSHHPAR |
| | MBPp9 | MBP 23 | HPARTAHYGSLPQKSH |
| | | MBP 24 | TAHYGSLPQKSHGRTQ |
| MBPp4 | MBPm10 | MBP 25 | GSLPQKSHGRTQDENP |
| | | MBP 26 | QKSHGRTQDENPVVHF |
| | MBPm11 | MBP 27 | GRTQDENPVVHFFKNI |
| | | MBP 28 | DENPVVHFFKNIVTPR |
| | MBPm12 | MBP 29 | VVHFFKNIVTPRTPPP |
| | | MBP 30 | FKNIVTPRTPPPSQGK |
| MBPp5 | MBPm13 | MBP 31 | VTPRTPPPSQGKGAEG |
| | | MBP 32 | TPPPSQGKGAEGQRPG |
| | MBPm14 | MBP 33 | SQGKGAEGQRPGFGYG |
| | | MBP 34 | GAEGQRPGFGYGGRAS |
| | MBPm15 | MBP 37 | GRASDYKSAHKGFKGV |
| | | MBP 38 | DYKSAHKGFKGVDAQG |
| MBPp6 | MBPm16 | MBP 39 | AHKGFKGVDAQGTLSK |
| | | MBP 40 | FKGVDAQGTLSKIFKL |
| | MBPm17 | MBP 41 | DAQGTLSKIFKLGGRD |
| | | MBP 42 | TLSKIFKLGGRDSRSG |
| | MBPm18 | MBP 43 | IFKLGGRDSRSGSPVA |
| | | MBP 44 | GGRDSRSGSPMARR |

MOG

| Mix | Pool | Seq ID | Sequence |
|---|---|---|---|
| MOGp1 | MOGm1 | MOG 1 | GQFRVIGPRHPIRALV |
| | | MOG 2 | VIGPRHPIRALVGDEV |
| | MOGm2 | MOG 3 | RHPIRALVGDEVELPC |
| | | MOG 4 | RALVGDEVELPCRISP |
| | MOGm3 | MOG 5 | GDEVELPCRISPGKNA |
| | | MOG 6 | ELPCRISPGKNATGME |
| MOGp2 | MOGm4 | MOG 7 | RISPGKNATGMEVGWY |
| | | MOG 8 | GKNATGMEVGWYRPPF |
| | MOGm5 | MOG 9 | TGMEVGWYRPPFSRVV |
| | | MOG 10 | VGWYRPFFSRVVHLYR |
| | MOGm6 | MOG 11 | RPPFSRVVHLYRNGKD |
| | | MOG 12 | SRVVHLYRNGKDQDGD |
| MOGp3 | MOGm7 | MOG 13 | HLYRNGKDQGDGGAPE |
| | | MOG 14 | NGKDQDGDDAPEYRGR |
| | MOGm8 | MOG 21 | VTLRIRNVRFSDEGGF |
| | | MOG 22 | IRNVRFSDEGGFTCFF |
| | MOGm9 | MOG 23 | RFSDEGGFTCFFRDHS |
| | | MOG 24 | EGGFTCFFRDHSYQEE |
| MOGp4 | MOGm10 | MOG 27 | YQEEAAMELKVEDPFY |
| | | MOG 28 | AAMELKVEDPFYWVSP |
| | MOGm11 | MOG 29 | LKVEDPFYWVSPGVLV |
| | | MOG 30 | DPFYWVSPGVLVLLAV |
| | MOGm12 | MOG 31 | WVSPGVLVLLAVLPVL |
| | | MOG 32 | GVLVLLAVLPVLLLQI |
| MOGp5 | MOGm13 | MOG 37 | FLCLQYRLIRGKLRAE |
| | | MOG 38 | LQYRLRGKLRAEIENL |
| | MOGm14 | MOG 39 | LRGKLRAEIENLHRTF |
| | | MOG 40 | LRAEIENLHRTFCPHF |
| | MOGm15 | MOG 41 | IENLHRTFDPHFLRVP |
| | | MOG 42 | HRTFDPHFLRVPCWKI |
| MOGp6 | MOGm16 | MOG 43 | DPHFLRVPCWKITLFV |
| | | MOG 44 | LRVPCWKITLPVIVPV |
| | MOGm17 | MOG 45 | CWKITLFVIVPVLGPL |
| | | MOG 46 | TLFVIVPVLGPLVALI |
| | MOGm18 | MOG 47 | IVPVLGPLVALIICYN |
| | | MOG 48 | LGPLVALIICYNWLHR |
| | MOGm19 | MOG 51 | WLHRRLAGQFLEELRN |
| | | MOG 52 | RLAGQFLEELRNFF |

PLP

| Pool | Mix | Seq ID | Sequence |
|---|---|---|---|
| PLPp1 | PLPm1 | PLP 1 | MGLLECCARCLVGAFF |
| | | PLP 2 | ECCARCLVGAPFASLV |
| | PLPm2 | PLP 7 | CFFGVALFCGCGHEAL |
| | | PLP 8 | VALFCGCGHEALTGTE |
| | PLPm3 | PLP 11 | TGTEKLIETYFSKNYQ |
| | | PLP 12 | KLIETYFSKNYQDYEY |
| PLPp2 | PLPm4 | PLP 21 | LYGALLLAEGFYTTGA |
| | | PLP 22 | LLLAEGFYTTGAVRQI |
| | PLPm5 | PLP 23 | EGFYTTGAVRQIFGDY |
| | | PLP 24 | TTGAVRQIFGDYKTTI |
| | PLPm6 | PLP 25 | VRQIFGDYKTTICGKG |
| | | PLP 26 | FGDYKTTICGKGLSAT |
| PLPp3 | PLPm7 | PLP 33 | QHQAHSLERVCHCLGK |
| | | PLP 34 | HSLERVCHCLGKWLGH |
| | PLPm8 | PLP 35 | RVCHCLGKWLGHPDKF |
| | | PLP 36 | CLGKWLGHPDKFVGIT |
| | PLPm9 | PLP 37 | WLGHPDKFVGITYALT |
| | | PLP 38 | PDKFVGITYALTVVWL |
| PLPp4 | PLPm10 | PLP 43 | CXAVPVYIYFNTWTTC |
| | | PLP 44 | PVYIYFNTWTTCQSIA |
| | PLPm11 | PLP 47 | QSIAFPSKTSASIGSL |
| | | PLP 48 | FPSKTSASIGSLCADA |
| | PLPm12 | PLP 49 | TSASIGSLCADARMYG |
| | | PLP 50 | IGSLCASARMYGVLPW |
| PLPp5 | PLPm13 | PLP 51 | CADARMYGVLPWNAFP |
| | | PLP 52 | RMYGVLPWNAFPGKVC |
| | PLPm14 | PLP 53 | VLPWNAFPGKVCGSNL |
| | | PLP 54 | NAPFGKVCGSNLLSIC |
| | PLPm15 | PLP 55 | GKVCGSNLLSICKTAE |
| | | PLP 56 | GSNLLSICKTAEFQMT |
| PLPp6 | PLPm16 | PLP 63 | ATLVSLLTFMIAATYN |
| | | PLP 64 | SLLTFMIAATYNFAVL |
| | PLPm17 | PLP 65 | FMIAATYNFAVLKLMG |
| | | PLP 66 | ATYNFAVLKLMGRGTK |
| | PLPm18 | PLP 67 | FAVLKLMGRGTKF |

1. A method of detecting an antigen specific T cell and identifying an immunostimulatory epitope to which the antigen specific T cell responds comprising
(a) priming in vitro at least one macrobulk culture of a sample comprising T cells from a subject with an epitope pool comprising one or more peptides, wherein each peptide in the epitope pool is a distinct fragment of an antigen;
(b) restimulating the macrobulk culture with the epitope pool for a period of time sufficient to allow for the detectable release of at least one activation cytokine by T cells specific for at least one of the peptides in the epitope pool; and
(c) detecting the absence or presence of the at least one activation cytokine in said macrobulk culture;
wherein the presence of the at least one activation cytokine in the culture detects a T cell specific for the antigen and identifies the fragment of the antigen as comprising an immunostimulatory epitope to which the antigen specific T cell responds.

2. The method of claim 1, wherein each epitope pool comprises at least two peptides, wherein each peptide shares a region of overlapping amino acid sequence identity with at least one other peptide in the epitope pool, the peptides in the epitope pool together span a contiguous region of the antigen, and wherein the presence of the at least one activation cytokine in the culture identifies the region spanned by the peptides in the epitope pool as comprising an immunostimulatory epitope to which the antigen specific T cell responds.

3. The method of claim 1, wherein each macrobulk culture comprises T cells at a density of 4.5×10⁵ cells/mL/mm³.

4. The method of claim 1, wherein the sample comprises peripheral blood mononuclear cells isolated from a patient.

5. The method of claim 4, wherein the patient has a disease and the antigen is associated with the disease.

6. The method of claim 5, wherein the disease is an autoimmune disorder and the antigen is an autoantigen associated with the autoimmune disorder.

7. The method of claim 6, wherein the autoimmune disorder is multiple sclerosis and the autoantigen is a myelin protein.

8. The method of claim 7, wherein the myelin protein is selected from the group consisting of myelin basic protein, proteolipid protein, myelin oligodendrocyte protein, and a combination thereof.

9. The method of claim 5, wherein the disease is a tumor and the antigen is a tumor associated antigen.

10. The method of claim 1, wherein the peptides are about 16 amino acids in length.

11. The method of claim 1, wherein each peptide pool has at least 2 different peptides.

12. The method of claim 11, wherein the region of overlapping amino acids between two different peptides is 12 amino acids in length.

13. The method of claim 1, wherein the activation cytokine is IFNγ.

14. The method of claim 1, wherein the activation cytokine is detected by ELISA.

15. The method of claim 1, wherein the epitope pool is from a library comprising at least two epitope pools, and wherein steps (a)-(c) are repeated for each epitope pool in the library.

16. The method of claim 15, wherein the library of epitope pools comprises peptides that span at least 50% of the antigen.

17. The method of claim 1, wherein the detecting step comprises detecting the absence or presence of two activation cytokines in said macrobulk culture; wherein the presence of both activation cytokines in the culture detects a T cell specific for the antigen and identifies the fragment of the antigen as comprising an immunostimulatory epitope to which the antigen specific T cell responds.

18. The method of claim 17, wherein the two activation cytokines are selected from the group consisting of (a) IFNγ and TNFα, (b) IFNγ and IL-6, and (c) TNFα and IL-6.

19. A method of making a composition for the treatment of a patient having a disease comprising
(a) detecting an antigen specific T cell in a sample from the patient and identifying an immunostimulatory epitope of an antigen associated with the disease and to which the antigen specific T cell responds according to the method of claim 1; and
(b) propagating T cells isolated from the patient with the identified immunostimulatory epitope.

20. The method of claim 19, wherein the disease is cancer and the antigen is a tumor associated antigen or a tumor specific antigen associated with the cancer.

21. The method of claim 20, further comprising as a last step the step of attenuating the propagated T cells.

22. The method of claim 21, wherein the disease is an autoimmune disorder and the antigen is an autoantigen associating with the autoimmune disorder.

23. A composition comprising T cells made according to the method of claim 19.

24. A method of treating a disease comprising administering the composition made according to the method of claim 19.
